(19) United States
(12) Patent Application Publication
(10) Pub. No.: US 2025/0134984 A1
(43) Pub. Date: May 1, 2025

(54) DEVELOPMENT OF MOSAIC VACCINES AGAINST FOOT AND MOUTH DISEASE VIRUS SEROTYPE ASIA

(71) Applicants: The United States of America, as represented by the Secretary of Agriculture, Washington, DC (US); Triad National Security, LLC, LOS ALAMOS, NM (US); KANSAS STATE UNIVERSITY RESEARCH FOUNDATION, Manhattan, KS (US)

(72) Inventors: AIDA E. RIEDER, WESTBROOK, CT (US); KATHERINE M. PFLAUM, MIDDLETOWN, CT (US); FAYNA C. DIAZ SAN SEGUNDO, BLUE POINT, NY (US); WILLIAM M. FISCHER, LOS ALAMOS, NM (US); IGNACIO FERNANDEZ SAINZ, OLD SAYBROOK, CT (US)

(21) Appl. No.: 18/924,027

(22) Filed: Oct. 23, 2024

Related U.S. Application Data
(60) Provisional application No. 63/593,277, filed on Oct. 26, 2023.

(51) Int. Cl. A61K 39/135; A61K 39/00; A61P 37/04; C07K 14/00; C12N 15/86

(52) U.S. Cl. CPC A61K 39/135; A61P 37/04; C07K 14/00; C12N 15/86; A61K 2039/552; A61K 2039/575; C12N 2770/32122; C12N 2770/32134; C12N 2770/32143; C12N 2770/32171

(57) ABSTRACT

Synthetic foot-and-mouth disease virus (FMDV) mosaic polypeptides, and nucleic acid molecules encoding the mosaic polypeptides, are described. When included as part of an FMDV genome, the mosaic polypeptides permit virus replication and assembly into FMDV particles. The mosaic polypeptide and nucleic acid compositions can be used to elicit immune responses that provide protection against a broad range of serotype Asia FMDV strains.

### CROSS-REFERENCE

The present application claims priority to U.S. Provisional Patent Application Ser. No. 63/593,277 filed Oct. 26, 2023, the content of which is expressly incorporated herein by reference.

### ACKNOWLEDGMENT OF GOVERNMENT SUPPORT

This invention was made with government support under IAA Contract No. 70RSAT18KPM000140, awarded by the U.S. Department of Homeland Security and 89233218CNA000001 awarded by the U.S. Department of Energy. The government has certain rights in the invention.

### SEQUENCE LISTING

The instant application contains a Sequence Listing XML required by 37 C.F.R. § 1.831 (a) which has been submitted in XML file format via the USPTO patent electronic filing system and is hereby incorporated by reference in its entirety. The XML file was created on Oct. 16, 2023 is named Sequence_Listing_0091_22.xml and has 19.0 KB bytes.

### FIELD OF INVENTION

This disclosure concerns synthetic foot-and-mouth disease virus (FMDV) polypeptides and nucleic acids and their use for inducing a broad immune response against serotype Asia FMDV. Recombinant viruses expressing these polypeptides are also disclosed, as are methods of using the viruses to induce immune responses to FMDV.

### BACKGROUND OF THE INVENTION

### Background

Foot-and-mouth disease (FMD) is one of the most highly contagious viral diseases of cloven-hoofed animals and it was the first official notifiable disease defined by the World Organization for Animal Health (WOAH). The virus can infect over 70 species of livestock and wild animals including cattle, swine, sheep, goat, and buffalo and is the single most important constraint to trade in live animals and animal products. Diseases induced by RNA viruses are difficult to control due to their inherently high mutation rate and the generation of antigenic variants that result in escape from immunity. The antigenic differences among them are critical to the degree of immunity induced against one another and influence their capacity to evade the immune response.

Foot-and-mouth disease virus (FMDV), a member of the Aphthovirus genus within the Picornaviridae family, is the causative agent of an economically important disease of cloven-hoofed animals, and the single most important constraint to trade in live animals and animal products. FMDV is a non-enveloped positive-single-stranded RNA virus of ˜8.5 Kb that occurs as seven distinct serotypes (A, O, C, SAT1, SAT2, SAT3 and Asia 1). Diversity of target viruses is a major problem for FMDV because a virus from different serotypes do not induce cross protection against each other after either infection or vaccination (Bachrach, H. L., Ann. Rev. Microbiol., (1968), 22:201-44; Domingo et al., Virus Res., (2003), 91:47-63). Within a serotype, distinct genetic and antigenic variants exist in different geographic regions and the emergence of new antigenic variant strains sometimes fail to induce efficient cross-protection against other viruses isolates from the same serotype. In addition, there are constant challenges due to the emergence of new antigenic variants. Developing vaccines with high efficacy against a broader antigenic spectrum of FMDV isolates will contribute to better FMD control and will be beneficial to the livestock smallholders in developing countries.

### SUMMARY OF THE INVENTION

The present disclosure provides, in one embodiment a synthetic polypeptide having an amino acid sequence at least 96% identical to SEQ ID NO: 2, SEQ ID NO: 4, SEQ ID NO: 6, or SEQ ID NO: 8. In specific embodiments, the synthetic polypeptide has the amino acid sequence of SEQ ID NO: 2, SEQ ID NO: 4, SEQ ID NO: 6, or SEQ ID NO: 8. Some embodiments of this composition also contain a pharmaceutically acceptable carrier. In other embodiments, a first and second synthetic polypeptide are present where the first synthetic polypeptide comprises an amino acid sequence at least 96% identical to SEQ ID NO: 2, and the second synthetic polypeptide comprising an amino acid sequence at least 96% identical to SEQ ID NO: 4; such two polypeptide compositions can optionally contain a pharmaceutically acceptable carrier and/or and adjuvant.

An additional embodiment disclosed herein is a recombinant foot-and-mouth disease virus (FMDV) comprising a synthetic polypeptide having an amino acid sequence at least 96% identical to SEQ ID NO: 2, SEQ ID NO: 4, SEQ ID NO: 6, or SEQ ID NO: 8. The recombinant FMDV of claim 8, comprising a synthetic polypeptide comprising the amino acid sequence of SEQ ID NO: 2, SEQ ID NO: 4, SEQ ID NO: 6, or SEQ ID NO: 8. In other embodiments, at least two recombinant FMDV are present in a mixture, where the first recombinant FMDV comprises a synthetic polypeptide at least 98% identical to the amino acid sequence of SEQ ID NO: 2, and the second recombinant FMDV comprises a synthetic polypeptide at least 96% identical to the amino acid sequence of SEQ ID NO: 4; such two-virus mixes can optionally also contain a pharmaceutically acceptable carrier and/or an adjuvant.

Further provided herein is an isolated nucleic acid molecule encoding a synthetic polypeptide at least 96% identical to SEQ ID NO: 2, SEQ ID NO: 4, SEQ ID NO: 6, or SEQ ID NO: 8. In specific embodiments, such isolated nucleic acids comprise the nucleotide sequence of SEQ ID NO: 1, SEQ ID NO: 3, SEQ ID NO: 5, or SEQ ID NO: 7. Also disclosed herein are vectors comprising any of these nucleic acids.

An additional embodiment provided herein, is a method of eliciting an immune response against serotype Asia foot-and-mouth disease virus (FMDV) in a subject, comprising administering to the subject a composition comprising the synthetic polypeptide at least 96% identical to SEQ ID NO: 2, SEQ ID NO: 4, SEQ ID NO: 6, or SEQ ID NO: 8, thereby eliciting an immune response to serotype Asia FMDV. In some embodiments of this method, the synthetic polypeptide comprises SEQ ID NO: 2, SEQ ID NO: 4, SEQ ID NO: 6, or SEQ ID NO: 8. In some embodiments, the administered composition further comprises a pharmaceutically acceptable carrier, an adjuvant, or both a pharmaceutically acceptable carrier and an adjuvant. In specific embodiments, two compositions are administered to a subject, wherein the first composition comprises a synthetic polypeptide at least 96% identical to the amino acid sequence of SEQ ID NO: 2 and the second composition comprises a second synthetic polypeptide at least 98% identical to the amino acid sequence of SEQ ID NO: 4. In some embodiments, the subject is a bovine (e.g., cow) or swine (e.g., pig).

### INCORPORATION BY REFERENCE

All publications, patents and patent applications mentioned in this specification are herein incorporated by reference to the same extent as if each individual publication, patent or patent application was specifically and individually indicated to be incorporated by reference.

### BRIEF DESCRIPTION OF THE DRAWINGS

The novel features of the invention are set forth with particularity in the claims. Features and advantages of the present invention are referred to in the following detailed description, and the accompanying drawings of which:

FIG. 1 provides graphical representation of predicted coverage conferred by Asia mosaic capsids generated by an algorithm as described in Fischer et al, Nat. Med., (2007), 13:100-106, or the related “Epigraph” method (Theiler & Korber, Nat. Med., (2018), 37 (2): 181-194) to generate synthetic high-coverage sequences. X axis: mean 9-mer coverage of the natural sequence target set. Y axis: the number of times a particular coverage was observed out of 1000 random selections from the target set. Paired thin and broad arrows in each panel mark the coverage values of a current natural-sequence vaccine strain (Asia1-Shamir, thin) and mosaic vaccine cocktails (thick). Upper panel: single-component conceptual vaccines (one natural isolate or mosaic); center panel: two-component vaccines; lower panel: three-component vaccines.

FIG. 2A, FIG. 2B, FIG. 2C, and FIG. 2D provide graphic representation of full-length clones and derivation of type Asia mosaic FMDV vaccines. The FMDV genome organization of full-length clones (FLC in FIG. 2A) and marker leaderless (FMD-LL3B3D in FIG. 2C) type Asia mosaic FMDV mutants. The two unique restriction endonuclease sites (Fsel and Nhel) used to clone the mosaic capsids into the backbone (VP4-VP1) are shown (▴). The basic FMDV genome organization is shown depicting the locations of proteins encoded by the viral open reading frame (ORF) and elements encoded in the 5′ and 3′ untranslated region (UTR). FIG. 2B and FIG. 2D show tissue-culture propagation and plaque morphology of infectious-nucleic-acid of type Asia mosaic FLC (FIG. 2B) and FMD-LL3B3D (FIG. 2D) constructs, respectively in BHK-21 cells and BHK-αVβ6 cells. Viral plaques (centers of viral replication) are seen as light spots on the darker background of uninfected cells in the plate. The plaque phenotypes of the FMDV-LL3B3D Asia mosaic viral constructs are shown on BHK-21 and BHK-21αVβ6 monolayers stained at 72 and 48 hours respectively.

FIG. 3A, FIG. 3B, FIG. 3C, FIG. 3D, and FIG. 3E provide one-step growth curves and Western blot analysis. Growth curves were conducted in BHK or BHK-21αVβ6 for full length (FLC) and FMDV-LL3B3D of type Asia mosaic FMDV vaccine candidates as compared with parental Asia1/Shamir WT virus at different times (1, 4 7, 24 and/or 48 hours post-infection): FLC+Asia mosaic in BHK-21 cells (FIG. 3A), FLC+Asia Mosaic in BHK-21αVβ6 (FIG. 3B), FMDV-LLV3B3D Asia mosaics in BHK-21 cells (FIG. 3C), FMDV-LLV3B3D Asia mosaics in BHK-21 αVβ6 cells (FIG. 3D). Samples obtained at indicated timepoints hours post infection were titrated in BHKαVβ6 cells stained with crystal violet at 48 hpi. Western Blot analysis of FMDV Capsid proteins extracted from BHK cells infected with the indicated virus (FIG. 3E). Following infection, cell lysates were prepared and viral proteins were separated by SDS-PAGE and visualized by Western blotting using either Mouse monoclonal anti-FMDV VP2 (F14 Kindly provided by Dr. Alfonso Clavijo), or cross-reactive anti-FMDV VP1 (6HC4 and 7CH3) were produced at Plum Island Foreign Animal Disease Center (Robertson et al, Virus Res., (1984), 1:489-500).

FIG. 4A, FIG. 4B, FIG. 4C, and FIG. 4D provide representation of data on biostability of parental and mosaic Asia FMD viruses. FMDV are acid sensitive and can be also easily disassemble at high temperatures. Measures of in vitro biophysical stability revealed that FMD-LL3B3D (FIG. 4A and FIG. 4B) and Full length (FIG. 4C and FIG. 4D) Asia Mosaic viruses (Asia Mosaics 2.1 (SEQ ID NO: 2) and 2.2 (SEQ ID NO: 4)) displayed higher stability to acidification within a pH range between 4.3 to 6.8 and temperatures around the 48-56° C. compared to corresponding parental type Asia1 strains (FMDV Asia Shamir (SH) or FMDV-LL3B3D Asia1 India [LL Asia IND]). Under the experimental conditions the mosaic Asia viruses incubated for 30 minutes in a physiological buffer within the range 4-45° C. they showed similar titers as parental viruses (FIG. 4B and FIG. 4D).

FIG. 5A, FIG. 5B, FIG. 5C, and FIG. 5D provide representation of viremia and viral shedding in vaccinated and control cows challenged with FMDV Asia1/Shamir. FMDV RNA detection in serum (FIG. 5A) and nasal swabs (FIG. 5C) was performed using quantitative real-time RT-PCR and is presented as Log 10 genome copy numbers (GCN)/ml. Data presented are averaged values from 3 animals and error bars indicate standard deviations. Transformation of Ct values into GCN/ml was performed by use of standard curves based on analysis of 10-fold dilutions of in vitro synthesized FMDV RNA. RNA copy detection level was 10³/mL. Viable FMDV detection in serum (FIG. 5B) and nasal swabs (FIG. 5D) was performed using plaque assays where indicated samples were titrated on BHK-αVβ6 monolayers under a tragacanth overlay and stained with crystal violet at 24 hpi. Data presented are averaged values from three animals and error bars indicate standard deviations.

FIG. 6 provides graphic representation of T-Cell IFNγ ELISpot Reponses. Post vaccination IFNγ response to FMDV Asia1/Shamir in all vaccination groups pre- and post-challenge. At each sampling time point, heparinized blood was collected from each cow and PBMC's were isolated and IFNγ ELIspots were conducted according to manufacturer's instructions. Each data point represents the average of 3 animals±standard deviation.

FIG. 7A, FIG. 7B, FIG. 7C, and FIG. 7D provide graphic representation of neutralizing antibody titers against FMDV vaccine strains (FIG. 7B, FIG. 7C, and FIG. 7D) and challenge strains (FIG. 7A) in serum samples collected weekly from the day of vaccination (−21 dpc) until +28 days post-challenge. End-point titers are expressed as the reciprocal of the highest dilution of serum the protects 50% of the wells from FMDV-induced CPE. Each data point represents the mean±Standard deviation of each treatment group.

FIG. 8 provides assessment of DIVA Diagnostic compatibility of animals vaccinated with FMD-LL3B3D vaccines before and after heterologous challenge with FMDV Asia1/Shamir using the PrioCheck Assay. Serum was collected before vaccination (0 DPV) and at 0 (21 DPV), 3- and 7-days post-challenge. Samples with a percent inhibition (PI) of 50 or less were considered negative while samples with a PI greater than 50 were considered positive as per manufacturer instructions. Each data point in the group mean±standard deviation (SD).

FIG. 9A, FIG. 9B, FIG. 9C, and FIG. 9D provide results of hematological and clinical disease analysis. The percent of lymphocyte (% LY) populations in individual vaccinated (FIG. 9B, FIG. 9C, and FIG. 9D) and unvaccinated control animals (FIG. 9A) and clinical scores. Whole blood was collected in EDTA tubes and a differential cell blood count were conducted using a Hema Vet 950FS (Drew Scientific), according to manufacturer's instructions. Clinical scores were evaluated as number of feet with detectable vesicles plus vesicles observed in the nose-mouth area other than the inoculation of the challenge (maximum score of 5). Each data point represents the mean±SD of each treatment group.

FIG. 10 provides representation of clinical temperatures of vaccinated cows after FMDV Asia1/Shamir challenge. Each data point represents the group mean±SD.

FIG. 11 provides representation of clinical scores. Following FMDV Asia1/Shamir challenge, pigs were evaluated for clinical scores for 7 days by determining the number of feet displaying FMD vesicles and the presence/absence of lesions in the mouth/snout. The maximum score considered was 16. Each point indicates the mean clinical score of each group±SD. All PBS control animals either died or were euthanized at 3 dpc.

FIG. 12 provides graphic representation of clinical temperatures. Clinical temperatures of vaccinated pigs after FMDV Asia1/Shamir challenge. Each data point represents the group mean±SD.

FIG. 13A, FIG. 13B, FIG. 13C, and FIG. 13D provide representation of clinical temperatures and clinical scores. Clinical temperatures and clinical scores after FMDV Asia1/Shamir challenge in control (FIG. 13A) and vaccinated (FIG. 13B, FIG. 13C, and FIG. 13D) pigs. Each data point represents the group mean±SD. All control animals were euthanized by 3 dpc.

FIG. 14A, FIG. 14B, FIG. 14C, and FIG. 14D provide results of hematological analysis. The percent of lymphocyte (% LY) populations in individual vaccinated (FIG. 14A, FIG. 14B, FIG. 14C) and unvaccinated (FIG. 14D) control pigs pre- and post-challenge and clinical scores. Whole blood was collected in EDTA tubes and a differential cell blood counts were conducted using a Hema Vet 950FS (Drew Scientific), according to manufacturer's instructions.

FIG. 15 provides representation of DIVA Diagnostic compatibility. Assessment of DIVA diagnostic compatibility of pigs vaccinated with FMDV-LL3B3D vaccines before and after heterologous challenge with FMDV Asia1/Shamir using the PrioCheck Assay. Serum was collected before vaccination (0 dpv) and at −1 (28 dpv), 3 and 7 dpc. Samples with a percent inhibition (PI) of 50 or less were considered negative while samples with a PI greater than 50 were considered positive as per manufacturer instructions. Each data point represents the value of an individual animal.

FIG. 16 provides representation of T-Cell IFNγ ELISpot Reponses. At each time point displayed above, freshly isolated porcine PBMCs were exposed to FMDV Asia1/Shamir ex vivo and assayed for T Cell IFNγ production via ELISpot (MabTech) according to manufacture instructions. Each data point represents the value of an individual animal. All control animals were euthanized by 3 dpc.

FIG. 17A, FIG. 17B, FIG. 17C, and FIG. 17D provide results of analysis of neutralizing titers. Neutralizing antibody titers against FMDV challenge strains (FIG. 17A, FIG. 17B), and vaccine strains (FIG. 17C, FIG. 17D) in serum samples collected weekly from the day of vaccination (−28 dpc) until +14 dpc. End-point titers are expressed as the reciprocal of the highest dilution of serum the protects 50% of the wells from FMDV-induced CPE. Each data point represents the mean±SD of each treatment group.

FIG. 18A and FIG. 18B provides graphic representation of Serum Viremia (viable virus) in vaccinated and control cows challenged with either FMDV 01/Manisa (FIG. 18A) or A/Iran/05 (FIG. 18B). Viable FMDV detection in serum was performed using plaque assays where indicated samples were titrated on BHK-αvβ6 monolayers under a tragacanth overlay and stained with crystal violet at 24 hpi. Data presented are averaged values from three animals and error bars indicate ±standard deviations.

FIG. 19A and FIG. 19B provides graphic representation of Viral shedding (viable virus) in vaccinated and control cows challenged FMDV O1/Manisa (FIG. 19A) or A/Iran/05 (FIG. 19B). Viable FMDV detection in nasal swabs was performed using plaque assays where indicated samples were titrated on BHK-αvβ6 monolayers under a tragacanth overlay and stained with crystal violet at 24 hpi. Data presented are averaged values from three animals and error bars indicate ±standard deviations.

FIG. 20A and FIG. 20B provides graphic representation of Serum Viremia (qRT-PCR) in vaccinated and control cows challenged with FMDV O1/Manisa (FIG. 20A) or A/Iran/05 (FIG. 20B). FMDV RNA detection in serum was performed using quantitative real-time RT-PCR and is presented as Log 10 genome copy numbers (GCN)/ml. Data presented are averaged values from 3 animals and error bars indicate standard deviations. Transformation of Ct values into GCN/ml was performed by use of standard curves based on analysis of 10-fold dilutions of in-vitro synthesized FMDV RNA. Data presented are averaged values from three animals and error bars indicate standard deviations.

FIG. 21A and FIG. 21B provides graphic representation of Viral shedding (qRT-PCR) in vaccinated and control cows challenged with FMDV 01/Manisa (FIG. 21A) or A/Iran/05 (FIG. 21B). FMDV RNA detection in nasal swabs was performed using quantitative real-time RT-PCR and is presented as Log 10 genome copy numbers (GCN)/ml. Data presented are averaged values from 3 animals and error bars indicate standard deviations. Transformation of Ct values into GCN/ml was performed by use of standard curves based on analysis of 10-fold dilutions of in-vitro synthesized FMDV RNA. Data presented are averaged values from three animals and error bars indicate standard deviations.

FIG. 22A and FIG. 22B provides graphic representation of Hematological analysis of vaccinated and unvaccinated animals. The percent of lymphocyte (% LY) (FIG. 22A) and number of lymphocytes (K/ul) (FIG. 22B) populations in individual vaccinated and unvaccinated control animals challenged with FMDV O1/Manisa. Whole blood was collected in EDTA tubes and differential blood counts were conducted using a Hema Vet 950FS (Drew Scientific), according to manufacturer's instructions. Each data point represents the group mean from three animals±Standard Deviation.

FIG. 23A and FIG. 23B provides graphic representation of hematological analysis of vaccinated and unvaccinated animals. The number of lymphocyte (% LY) (FIG. 23A) and number of lymphocytes (K/ul) (FIG. 23B) populations in individual vaccinated and unvaccinated control animals challenged with FMDV A/Iran/05. Whole blood was collected in EDTA tubes and differential blood counts were conducted using a Hema Vet 950FS (Drew Scientific), according to manufacturer's instructions. Each data point represents the group mean from three animals±Standard Deviation.

FIG. 24A and FIG. 24B provide graphic representation of neutralizing antibody titers against wild type FMDV Asia Mosaic 2.1 (FIG. 24A) and FMDV Asia Shamir (FIG. 24B) strains in serum samples collected weekly from the day of vaccination (−28 dpc) until +14 dpc. End-point titers are expressed as the reciprocal of the highest dilution of serum the protects 50% of the wells from FMDV-induced CPE. Each data point represents the mean±SD of each treatment group.

### DETAILED DESCRIPTION OF THE INVENTION

Provided herein is the development of full-length mosaic constructs for serotype Asia1, a serotype that has caused great devastation in Asia in recent years (Brito et al, Sci. Reports, (2018), 8:6472), and the serotype Asia in a low virulence and negative antigenic (marker) backbone (FMD-LL3B3D—U.S. Pat. No. 8,765,141). These vaccine viruses, as a single mosaic vaccine or in combinations of two mosaic cocktails, have potential for the delivery of an improved vaccine platform with broader coverage, of interest to FMD endemic and disease-free regions. An FMD-LL3B3D Mosaic Asia virus vaccine efficacy study was conducted in both cattle and swine that were challenged with heterologous virus. In addition, the in vitro growth characteristics and biophysical- and thermo-stability of the newly developed vaccine viruses demonstrated increase stability compared to parental strains. The results indicate that immunization of cattle with the chemically inactivated FMD-LL3B3D platform containing polyvalent Asia Mosaic capsids provided 100% protection with no clinical signs of disease after heterologous challenge with wild-type (WT) viruses. These attenuated, antigenically negatively marked viruses provide a safe alternative to virulent strains for FMD vaccine manufacturing with potentials to increase breath of coverage within type Asia strains from different global FMD pools.

Disclosed herein are synthetic FMDV mosaic proteins that have greater coverage of potential T-cell epitopes than do naturally occurring FMDV proteins. The synthetic FMDV mosaic polypeptides incorporate natural virus variability and include common FMDV subsequences but exclude rare FMDV subsequences. When included as part of an FMDV genome, the mosaic polypeptides permit viral replication and virus assembly into virus particles that are highly similar or identical in structure to native FMDV particles; however, the replacement of rare potential epitopes by common ones leads to in vivo immune responses to a wider range of FMDV strains than vaccines based on natural sequences alone. The mosaic polypeptide and nucleic acid compositions disclosed herein can be used to elicit immune responses that provide protection against a broad range of serotype A FMDV strains. Specific mosaic peptides against serotype Asia FMDV are provided as SEQ ID NO: 2, SEQ ID NO: 4, SEQ ID NO: 6, and SEQ ID NO: 8.

Preferred embodiments of the present invention are shown and described herein. It will be obvious to those skilled in the art that such embodiments are provided by way of example only. Numerous variations, changes, and substitutions will occur to those skilled in the art without departing from the invention. Various alternatives to the embodiments of the invention described herein may be employed in practicing the invention. It is intended that the included claims define the scope of the invention and that methods and structures within the scope of these claims and their equivalents are covered thereby.

Technical and scientific terms used herein have the meanings commonly understood by one of ordinary skill in the art to which the instant invention pertains, unless otherwise defined. Reference is made herein to various materials and methodologies known to those of skill in the art. Standard reference works setting forth the general principles of recombinant DNA technology include Sambrook et al., “Molecular Cloning: A Laboratory Manual”, 2d ed., Cold Spring Harbor Laboratory Press, Plainview, N.Y., 1989; Kaufman et al., eds., “Handbook of Molecular and Cellular Methods in Biology and Medicine”, CRC Press, Boca Raton, 1995; and McPherson, ed., “Directed Mutagenesis: A Practical Approach”, IRL Press, Oxford, 1991. Standard reference literature teaching general methodologies and principles of fungal genetics useful for selected aspects of the invention include Sherman et al. “Laboratory Course Manual Methods in Yeast Genetics”, Cold Spring Harbor Laboratory, Cold Spring Harbor, N.Y., 1986 and Guthrie et al., “Guide to Yeast Genetics and Molecular Biology”, Academic, New York, 1991.

Any suitable materials and/or methods known to those of skill can be utilized in carrying out the instant invention. Materials and/or methods for practicing the instant invention are described. Materials, reagents and the like to which reference is made in the following description and examples are obtainable from commercial sources, unless otherwise noted.

As used in the specification and claims, use of the singular “a”, “an”, and “the” include plural references unless the context clearly dictates otherwise.

The terms isolated, purified, or biologically pure as used herein, refer to material that is substantially or essentially free from components that normally accompany the referenced material in its native state.

The term “about” is defined as plus or minus ten percent of a recited value. For example, about 1.0 g means 0.9 g to 1.1 g and all values within that range, whether specifically stated or not.

The term “a nucleic acid consisting essentially of”, and grammatical variations thereof, means nucleic acids that differ from a reference nucleic acid sequence by 20 or fewer nucleic acid residues and also perform the function of the reference nucleic acid sequence. Such variants include sequences which are shorter or longer than the reference nucleic acid sequence, have different residues at particular positions, or a combination thereof.

The term “adjuvant”, as used herein refers to a substance or vehicle that non-specifically enhances the immune response to an antigen. Adjuvants can include a suspension of minerals (alum, aluminum hydroxide, or phosphate) on which antigen is adsorbed; or water-in-oil emulsion in which antigen solution is emulsified in mineral oil (for example, Freund's incomplete adjuvant), sometimes with the inclusion of killed mycobacteria (Freund's complete adjuvant) to further enhance antigenicity. Immunostimulatory oligonucleotides (such as those including a CpG motif) can also be used as adjuvants (for example, see U.S. Pat. Nos. 6,194,388; 6,207,646; 6,214,806; 6,218,371; 6,239,116; 6,339,068; 6,406,705; and 6,429,199). Adjuvants also include biological molecules, such as costimulatory molecules. Exemplary biological adjuvants include IL-2, RANTES, GM-CSF, TNF-α, IFN-γ, G-CSF, LFA-3, CD72, B7-1, B7-2, OX-40L and 41 BBL.

The term “administer” or “administration” is to provide or give a subject an agent, such as a therapeutic agent (e.g. a recombinant virus), by any effective route. Exemplary routes of administration include, but are not limited to, injection (such as subcutaneous, intramuscular, intradermal, intraperitoneal, and intravenous), oral, intraductal, sublingual, rectal, transdermal, intranasal, vaginal and inhalation routes.

As used herein, the term “immune response” and grammatical variations thereof refers to a response of a cell of the immune system, such as a B-cell, T-cell, macrophage or polymorphonucleocyte, to a stimulus such as an antigen or vaccine. An immune response can include any cell of the body involved in a host defense response, including for example, an epithelial cell that secretes an interferon or a cytokine. An immune response includes, but is not limited to, an innate immune response or inflammation. As used herein, a protective immune response refers to an immune response that protects a subject from infection (prevents infection or prevents the development of disease associated with infection). Methods of measuring immune responses are well known in the art and include, for example, measuring proliferation and/or activity of lymphocytes (such as B or T cells), secretion of cytokines or chemokines, inflammation, antibody production and the like.

The term “immunogen” refers to a compound, composition, or substance which is capable, under appropriate conditions, of stimulating an immune response, such as the production of antibodies or a T-cell response in an animal, including compositions that are injected or absorbed into an animal.

The pharmaceutically acceptable carriers (vehicles) useful in this disclosure are conventional. Remington's Pharmaceutical Sciences, by E. W. Martin, Mack Publishing Co., Easton, PA, 15th Edition (1975), describes compositions and formulations suitable for pharmaceutical delivery of one or more therapeutic compounds, molecules or agents (e.g. a mosaic polypeptide or recombinant virus disclosed herein). A suitable carrier can be determined by one skilled in the art. In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (for example, powder, pill, tablet, or capsule forms), conventional non-toxic solid carriers can include, for example, pharmaceutical grades of mannitol, lactose, starch, or magnesium stearate. In addition to biologically neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

“Polypeptide”, “peptide” and “protein” refers to a polymer in which the monomers are amino acid residues which are joined together through amide bonds. When the amino acids are alpha-amino acids, either the L-optical isomer or the D-optical isomer can be used. The terms “polypeptide,” “peptide” and “protein” are used interchangeably herein. These terms apply to amino acid polymers in which one or more amino acid residue is an artificial chemical mimetic of a corresponding naturally occurring amino acid, as well as to naturally occurring amino acid polymers and non-naturally occurring amino acid polymers. The term “residue” or “amino acid residue” includes reference to an amino acid that is incorporated into a protein, polypeptide, or peptide.

A conservative substitution in a polypeptide is a substitution of one amino acid residue in a protein sequence for a different amino acid residue having similar biochemical properties. Typically, conservative substitutions have little to no impact on the activity of a resulting polypeptide. For example, a protein or peptide including one or more conservative substitutions (for example no more than 1, 2, 3, 4 or 5 substitutions) retains the structure and function of the wild-type protein or peptide. A polypeptide can be produced to contain one or more conservative substitutions by manipulating the nucleotide sequence that encodes that polypeptide using, for example, standard procedures such as site-directed mutagenesis or PCR. In one example, such variants can be readily selected by testing antibody cross-reactivity or its ability to induce an immune response. Conservative substitutions are well known in the art.

Conservative substitutions generally maintain (a) the structure of the polypeptide backbone in the area of the substitution, for example, as a sheet or helical conformation, (b) the charge or hydrophobicity of the molecule at the target site, or (c) the bulk of the side chain.

The substitutions which in general are expected to produce the greatest changes in protein properties will be non-conservative, for instance changes in which (a) a hydrophilic residue, for example, seryl or threonyl, is substituted for (or by) a hydrophobic residue, for example, leucyl, isoleucyl, phenylalanyl, valyl or alanyl; (b) a cysteine or proline is substituted for (or by) any other residue; (c) a residue having an electropositive side chain, for example, lysyl, arginyl, or histadyl, is substituted for (or by) an electronegative residue, for example, glutamyl or aspartyl; or (d) a residue having a bulky side chain, for example, phenylalanine, is substituted for (or by) one not having a side chain, for example, glycine.

“Preventing” a disease refers to inhibiting the full development of a disease. “Treating” refers to a therapeutic intervention that ameliorates a sign or symptom of a disease or pathological condition after it has begun to develop. “Ameliorating” refers to the reduction in the number or severity of signs or symptoms of a disease.

A “recombinant” nucleic acid molecule, protein or virus is one that has a sequence that is not naturally occurring or has a sequence that is made by an artificial combination of two otherwise separated segments of sequence. This artificial combination can be accomplished by chemical synthesis or by the artificial manipulation of isolated segments of nucleic acid molecules, such as by genetic engineering techniques. The term “recombinant” also includes nucleic acids, proteins and viruses that have been altered solely by addition, substitution, or deletion of a portion of the natural nucleic acid molecule, protein or virus.

For the purpose of this invention, the “sequence identity” of two related nucleotide or amino acid sequences, expressed as a percentage, refers to the number of positions in the two optimally aligned sequences which have identical residues (×100) divided by the number of positions compared. A gap, i.e., a position in an alignment where a residue is present in one sequence but not in the other is regarded as a position with non-identical residues. The alignment of the two sequences is performed by the Needleman and Wunsch algorithm (Needleman and Wunsch, J Mol Biol, (1970) 48:3, 443-53). A computer-assisted sequence alignment can be conveniently performed using a standard software program such as GAP which is part of the Wisconsin Package Version 10.1 (Genetics Computer Group, Madison, Wisconsin, USA) using the default scoring matrix with a gap creation penalty of 50 and a gap extension penalty of 3.

The phrase “high percent identical” or “high percent identity”, and grammatical variations thereof in the context of two polynucleotides or polypeptides, refers to two or more sequences or sub-sequences that have at least about 80%, identity, at least about 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% nucleotide or amino acid identity, when compared and aligned for maximum correspondence, as measured using a sequence comparison algorithm or by visual inspection. In one exemplary embodiment, the sequences are high percent identical over the entire length of the polynucleotide or polypeptide sequences.

The term “subject” refers to a living multi-cellular vertebrate organism, a category that includes human and non-human mammals. In some embodiments herein, the subject is a cloven-footed animal, such as, but not limited to, a cow, pig, sheep, goat, deer, antelope, water buffalo or bison.

The term “vaccine” refers to a preparation of immunogenic material capable of stimulating an immune response, administered for the prevention, amelioration, or treatment of disease, such as an infectious disease. The immunogenic material may include, for example, attenuated or killed microorganisms (such as attenuated viruses), or antigenic proteins, peptides or DNA derived from an infectious microorganism. Vaccines may elicit both prophylactic (preventative) and therapeutic responses. Methods of administration vary according to the vaccine, but may include inoculation, ingestion, inhalation or other forms of administration. Inoculations can be delivered by any of a number of routes, including parenteral, such as intravenous, subcutaneous or intramuscular. Vaccines may be administered with an adjuvant to boost the immune response.

### Mosaic Peptides

The mosaic proteins described herein resemble natural proteins and have been designed to maximize the coverage of potential T-cell epitopes for a viral population (Fischer et al, Nat. Med., (2007), 13 (1): 100-106; Barouch et al, Nat. Med., (2010), 16 (3): 319-323). Humoral responses against FMDV (development of virus neutralizing antibodies) have historically been relied upon as a correlate of protection, however it has been shown that this is not always the case (Mccullough et al, J. Virol., (1992), 66 (4): 1835; Sobrino et al, Vet. Res., (2001), 32 (1): 1-30). Optimization for potential T-cell epitopes in this case means simply optimization for conserved linear amino-acid sequence, which is likely to preserve many antibody epitopes as well; vaccination with mosaic immunogens has been demonstrated to induce neutralizing antibodies directed towards rabies glycoprotein (Stading et al, PLOS Negl. Trop. Dis., (2017), 11 (10): e0005958) and protective antibodies to HIV-1 Env (Barouch, D. H., New Engl. J. Med., (2013), 369 (22): 2073-2076). Because a balanced immune response plays a role in protection from FMDV infection (Becker, Y., Virus Genes, (1994), 8 (3): 199-214; Sobrino et al, supra), FMDV-directed T-cell responses have been assessed in terms of IFNγ production. In vitro IFNγ responses have been measured in different capacities in FMD research from ELISA to T cell specific population assessment, MHC analysis, in vitro stimulation with inactivated FMD virus, or peptide pools using flow cytometry, ELISA and occasionally ELISpot assays (Zhang et al, Arch. Virol., (2002), 147 (11): 2157-2167; Parida et al, Vaccine, (2006), 24 (7): 964-969; Guzman et al, J. Virol., (2010), 84 (23): 12375; Toka et al, J. Immunol., (2011), 186 (8): 4853; Oh et al, PLOS One, (2012), 7 (9): e44365; Carr et al, J. Gen. Virol., (2013), 94 (Pt 1): 97-107; Bucafusco et al, Virol., (2015), 476:11-18; Sharma et al, Microb. Pathogen., (2018), 125:20-25). IFNγ has been shown to respond specifically against FMDV (Oh et al, supra; Bucafusco et al, supra; Sharma et al, supra).

The integrity and biophysical stability of FMD virus particles has been correlated with the capacity of these viruses to induce a protective immune response in susceptible species (Doel & Baccarini, Arch. Virol., (1981), 70 (1): 21-32; López-Argüello et al, J. Virol., (2019), 93 (10): e02293-02218). Though a certain amount of stability is needed to survive environments not naturally conducive to viral survival, at the same time, viruses (Doel & Chong, Arch. Virol., (1982), 73 (2): 185-191; Mateo et al, J. Virol., (2008), 82 (24): 12232-40; Rincón et al, Structure, (2014), 22 (11): 1560-1570) may need some instability to infect and propagate in their host environment (López-Argüello et al, supra).

Disclosed herein are synthetic FMDV mosaic polypeptides that have greater T-cell epitope coverage than naturally occurring FMDV polypeptides. When included as part of an FMDV genome, the mosaic polypeptides permit viral replication and virus assembly into structures that are highly similar or identical to native FMDV particles. The mosaic polypeptide and nucleic acid compositions disclosed herein can be used to elicit immune responses that provide protection against a broad range of serotype A FMDV strains.

In specific embodiments provided herein, FMDV polypeptides have an amino acid sequence at least 80%, at least 85%, at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% identical to mosaic polypeptide Asia 2.1 capsid (FLC) (SEQ ID NO: 2), mosaic polypeptide Asia 2.2 capsid (FLC) (SEQ ID NO: 4), mosaic polypeptide Asia2.1 capsid (FMD-LL3B3D) (SEQ ID NO: 6), or mosaic polypeptide Asia 2.2 capsid (FMD-LL3B3D) (SEQ ID NO: 8). In some embodiments, the synthetic FMDV polypeptide includes the amino acid sequence of SEQ ID NO: 2, SEQ ID NO: 4, SEQ ID NO: 6, or SEQ ID NO: 8. In specific examples, the synthetic FMDV polypeptide consists of the amino acid sequence of SEQ ID NO: 2, SEQ ID NO: 4, SEQ ID NO: 6, or SEQ ID NO: 8.

Recombinant FMDV that include mosaic polypeptides are also provided herein. In some embodiments, the recombinant FMDV includes a synthetic FMDV polypeptide having an amino acid sequence at least 80%, at least 85%, at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% identical to mosaic polypeptide Asia 2.1 capsid (FLC) (SEQ ID NO: 2), mosaic polypeptide Asia 2.2 capsid (FLC) (SEQ ID NO: 4), mosaic polypeptide Asia2.1 capsid (FMD-LL3B3D) (SEQ ID NO: 6), or mosaic polypeptide Asia 2.2 capsid (FMD-LL3B3D) (SEQ ID NO: 8). In some embodiments, the recombinant FMDV includes a synthetic polypeptide comprising the amino acid sequence of SEQ ID NO: 2, SEQ ID NO: 4, SEQ ID NO: 6, or SEQ ID NO: 8. In specific examples, the recombinant FMDV includes a synthetic FMDV polypeptide consisting of the amino acid sequence of SEQ ID NO: 2, SEQ ID NO: 4, SEQ ID NO: 6, or SEQ ID NO: 8.

Further provided herein are nucleic acid molecules encoding mosaic FMDV polypeptides. In some embodiments, the nucleic acid encodes a synthetic FMDV polypeptide having an amino acid sequence at least 80%, at least 85%, at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% identical to mosaic polypeptide Asia 2.1 capsid (FLC) (SEQ ID NO: 2), mosaic polypeptide Asia 2.2 capsid (FLC) (SEQ ID NO: 4), mosaic polypeptide Asia2.1 capsid (FMD-LL3B3D) (SEQ ID NO: 6), or mosaic polypeptide Asia 2.2 capsid (FMD-LL3B3D) (SEQ ID NO: 8). In some embodiments, the nucleic acid molecule encodes a synthetic FMDV polypeptide comprising the amino acid sequence of SEQ ID NO: 2, SEQ ID NO: 4, SEQ ID NO: 6, or SEQ ID NO: 8. In specific examples, the nucleic acid molecule encodes a synthetic FMDV polypeptide consisting of the amino acid sequence of SEQ ID NO: 2, SEQ ID NO: 4, SEQ ID NO: 6, or SEQ ID NO: 8. In some embodiments, the nucleic acid molecule has a nucleotide sequence at least 80%, at least 85%, at least 90%, at least 95%, at least 96%, at least 97%, at least 98% or at least 99% identical to SEQ ID NO: 1, SEQ ID NO: 3, SEQ ID NO: 5, or SEQ ID NO: 7. In some examples, the nucleic acid molecule has a nucleotide sequence comprising SEQ ID NO: 1, SEQ ID NO: 3, SEQ ID NO: 5, or SEQ ID NO: 7. In particular non-limiting examples, the nucleic acid molecule has a nucleotide sequence consisting of SEQ ID NO: 1, SEQ ID NO: 3, SEQ ID NO: 5, or SEQ ID NO: 7.

Vectors comprising the mosaic FMDV polypeptide-encoding nucleic acid molecules are also provided by the present disclosure. In some embodiments, the vector further includes coding sequences for other native or recombinant proteins, whereupon transfection of the vector into a permissive host cell, infectious FMDV is produced.

Also provided herein are compositions that include at least one mosaic FMDV polypeptide, at least one recombinant FMDV, or at least one mosaic FMDV polypeptide encoding nucleic acid or at least one vector disclosed herein.

In some embodiments, provided is a composition that includes a mosaic FMDV polypeptide having an amino acid sequence at least 80%, at least 85%, at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% identical to mosaic polypeptide Asia 2.1 capsid (FLC) (SEQ ID NO: 2), mosaic polypeptide Asia 2.2 capsid (FLC) (SEQ ID NO: 4), mosaic polypeptide Asia2.1 capsid (FMD-LL3B3D) (SEQ ID NO: 6), or mosaic polypeptide Asia 2.2 capsid (FMD-LL3B3D) (SEQ ID NO: 8), and a pharmaceutically acceptable carrier.

In some examples, the composition includes one or more mosaic FMDV polypeptides comprising or consisting of at least 80%, at least 85%, at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% identical to mosaic polypeptide Asia 2.1 capsid (FLC) (SEQ ID NO: 2), mosaic polypeptide Asia 2.2 capsid (FLC) (SEQ ID NO: 4), mosaic polypeptide Asia2.1 capsid (FMD-LL3B3D) (SEQ ID NO: 6), or mosaic polypeptide Asia 2.2 capsid (FMD-LL3B3D) (SEQ ID NO: 8). Any of these compositions can further comprise a pharmaceutically acceptable carrier.

Further provided herein are compositions that include a vector that includes a mosaic FMDV polypeptide-encoding nucleic acid molecule disclosed herein. In some embodiments, the composition includes a vector comprising a nucleic acid molecule having at least 80%, at least 85%, at least 90%, at least 95%, at least 96%, at least 97%, at least 98% or at least 99% identity to SEQ ID NO: 1, SEQ ID NO: 3, SEQ ID NO: 5, or SEQ ID NO: 7. Such compositions can further comprise a pharmaceutically acceptable carrier. In some examples, the composition includes a first vector comprising a nucleic acid molecule having at least 80%, at least 85%, at least 90%, at least 95%, at least 96%, at least 97%, at least 98% or at least 99% identity to SEQ ID NO: 1, SEQ ID NO: 3, SEQ ID NO: 5, or SEQ ID NO: 7, and a second vector comprising a nucleic acid molecule having at least 80%, at least 85%, at least 90%, at least 95%, at least 96%, at least 97%, at least 98% or at least 99% identity to SEQ ID NO: 1, SEQ ID NO: 3, SEQ ID NO: 5, or SEQ ID NO: 7, where the second vector has a different sequence to the first vector. Such compositions can also include a pharmaceutically acceptable carrier.

Any of the compositions provided herein can optionally include an adjuvant. Any adjuvant known in the art, or later developed, can be selected by the skilled artisan.

Further provided herein are methods of eliciting an immune response against serotype Asia FMDV in a subject. In some embodiments, the method includes administering to the subject a synthetic FMDV mosaic polypeptide, a recombinant FMDV, a nucleic acid molecule, a vector, or a composition disclosed herein. In some examples, the subject is a cow.

Also provided herein are methods of immunizing a subject against serotype Asia FMDV. In some embodiments, the method includes administering to the subject a synthetic FMDV mosaic polypeptide, a recombinant FMDV, a nucleic acid molecule, a vector, or a composition disclosed herein. In some examples in which the recombinant FMDV is administered, the recombinant FMDV is inactivated (such as with BEI) prior to administration. In some embodiments of the methods provided herein, the subject is a cloven-footed animal. In some examples, the cloven-footed animal is a cow, pig, sheep, goat, deer, antelope, water buffalo or bison.

### Administration of Mosaic FMDV Vaccine Compositions

The FMDV mosaic polypeptide and polynucleotide compositions described herein can be administered to a subject using any suitable delivery means. For example, FMDV polynucleotides or polypeptides can be administered parenterally, by injection, subcutaneously, intramuscularly, transdermally or transcutaneously. Certain adjuvants, for example LTK63, LTR72 or PLG formulations, can be administered intranasally or orally. Additional formulations that are suitable for other modes of administration include suppositories. For suppositories, traditional binders and carriers can include, for example, polyalkylene glycols or triglycerides; such suppositories can be formed from mixtures containing the active ingredient in the range of 0.5% to 10%, such as 1%-2%. Other oral formulations include such normally employed excipients as, for example, pharmaceutical grades of mannitol, lactose, starch, magnesium stearate, sodium saccharine, cellulose, magnesium carbonate, and the like. These compositions take the form of solutions, suspensions, tablets, pills, capsules, sustained release formulations or powders and contain 10%-95% of active ingredient, such as 25%-70%.

The FMDV mosaic vaccines disclosed herein can be prepared as injectables, either as liquid solutions or suspensions. Solid forms suitable for solution or suspension in liquid prior to injection may also be prepared. Such preparations can also be emulsified or encapsulated in liposomes. In some instances, the vaccine also includes a pharmaceutically acceptable carrier. Pharmaceutically acceptable carriers are well known to those in the art, and include without limitation large, slowly metabolized macromolecules, such as proteins, polysaccharides, functionalized sepharose, agarose, cellulose, cellulose beads and the like, polylactic acids, polyglycolic acids, polymeric amino acids such as polyglutamic acid, polylysine, and the like.

The FMDV mosaic vaccines disclosed herein can be formulated into an immunogenic compound as neutral or salt forms. Pharmaceutically acceptable salts include the acid addition salts (formed with free amino groups of the peptide) and those formed with inorganic acids such as, for example, hydrochloric or phosphoric acids, or organic acids such as acetic, oxalic, tartaric, maleic, and the like. Salts formed with the free carboxyl groups can also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

Vaccine compositions can also contain liquids or excipients, such as water, saline, glycerol, dextrose, ethanol, or the like, singly or in combination, as well as substances such as wetting agents, emulsifying agents, or pH buffering agents. Liposomes can also be used as a carrier for a composition disclosed herein.

Various co-stimulatory molecules can be included in the vaccine preparation or delivery protocol. These molecules can improve immunogen presentation to lymphocytes and include such proteins as B7-1 or B7-2, and cytokines such as GM-CSF, IL-2, and IL-12. Optionally, adjuvants can also be included in a composition. Various adjuvants may be used, including (1) aluminum salts (alum), such as aluminum hydroxide, aluminum phosphate, or aluminum sulfate; (2) oil-in-water emulsion formulations (with or without other specific immunostimulating agents such as muramyl peptides or bacterial cell wall components); (3) saponin adjuvants, or particles generated therefrom such as ISCOMs (immunostimulating complexes); (4) Complete Freund's Adjuvant (CFA) and Incomplete Freund's Adjuvant (IFA); (5) cytokines, such as interleukins (for example, IL-1, IL-2, IL-4, IL-5, IL-6, IL-7, or IL-12), interferons (for example, gamma interferon), macrophage colony stimulating factor (M-CSF), or tumor necrosis factor (TNF); (6) detoxified mutants of a bacterial ADP-ribosylating toxin such as a cholera toxin (CT), a pertussis toxin (PT), or an E. coli heat-labile toxin (LT); (7) other substances that act as immunostimulating agents to enhance the effectiveness of the composition; and (8) microparticles with adsorbed macromolecules.

The FMDV mosaic vaccine compositions disclosed herein can be administered in a manner compatible with the dosage formulation, and in such amount as will be prophylactically and/or therapeutically effective. The quantity to be administered depends on the subject to be treated, the capacity of the subject's immune system, and the degree of protection desired. Precise amounts of active ingredient required to be administered may depend on the judgment of the practitioner and can be specific to each subject.

Vaccine formulations can be introduced in a single dose schedule, or in a multiple dose schedule. A multiple dose schedule is one in which a primary course of vaccination can be with 1-10 separate doses, followed by other doses given at subsequent time intervals required to maintain and or reinforce the immune response, for example, at 1-4 months for a second dose, and if needed, a subsequent dose(s) after several months.

The course of administration can include polynucleotides and polypeptides, together or sequentially (for example, priming with a polynucleotide composition and boosting with a polypeptide composition). The dosage regimen will also, at least in part, be determined by the need of the individual and be dependent upon the judgment of the practitioner.

Nucleic acid molecules and vectors comprising expressible polynucleotides encoding FMDV mosaic proteins can be formulated and utilized as DNA vaccine preparations. Such FMDV mosaic DNA vaccines can be used to activate FMDV-specific T cells, using standard gene delivery protocols. Methods for gene delivery are known in the art (see, for example, U.S. Pat. Nos. 5,399,346, 5,580,859, 5,589,466, incorporated by reference herein in their entireties). Genes can be delivered either directly to the vertebrate subject or, alternatively, delivered ex vivo, to cells derived from the subject and the cells reimplanted in the subject. For example, the constructs can be delivered as plasmid DNA, or viral vector DNA.

DNA vaccines can be introduced by a number of different methods, including by injection of DNA in saline, using a standard hypodermic needle. Injection in saline is typically conducted intramuscularly in skeletal muscle, or intradermally, with DNA being delivered to the extracellular spaces. This can be assisted by electroporation, by temporarily damaging muscle fibers with myotoxins such as bupivacaine or by using hypertonic solutions of saline or sucrose. Immune responses to this method of delivery can be affected by many factors, including needle type, needle alignment, speed of injection, volume of injection, muscle type, and age, sex and physiological condition of the individual being injected.

The method of delivery determines the dose of DNA required to raise an effective immune response. Saline injections require variable amounts of DNA, from 10 μg-1 mg, whereas gene gun deliveries require 100 to 1000 times less DNA than intramuscular saline injection to raise an effective immune response. Generally, 0.2 μg to 20 μg are required, although quantities as low as 16 ng have been utilized. Saline injections require more DNA because the DNA is delivered to the extracellular spaces of the target tissue (typically, muscle tissue), where physical barriers such as the basal lamina and large amounts of connective tissue must be overcome before it is taken up by the cells, while gene gun deliveries bombard DNA directly into the cells.

FMDV mosaic nucleic acid vaccines can be packaged in liposomes prior to delivery to cells. Lipid encapsulation is generally accomplished using liposomes which are able to stably bind or entrap and retain nucleic acid. The ratio of condensed DNA to lipid preparation can vary but will generally be around 1:1 (mg DNA:micromoles lipid), or more of lipid. Liposomal preparations for use with the disclosed FMDV vaccines include cationic (positively charged), anionic (negatively charged) and neutral preparations.

The FMDV mosaic nucleic acid vaccines can also be encapsulated, adsorbed to, or associated with, particulate carriers. Such carriers present multiple copies of a selected molecule to the immune system and promote trapping and retention of molecules in local lymph nodes. The particles can be phagocytosed by macrophages and can enhance antigen presentation through cytokine release. Examples of particulate carriers include those derived from polymethyl methacrylate polymers, as well as microparticles derived from poly(lactides) and poly(lactide-co-glycolides), known as PLG (see, for example, Jeffery et al., Pharm Res 10:362-368, 1993).

### Assessing Efficacy of FMDV Mosaic Vaccines

The ability of a particular mosaic protein or vaccine composition to stimulate a cell-mediated immunological response can be determined by any one of a number of assays, such as by lymphoproliferation (lymphocyte activation) assays, cytotoxic T lymphocyte (CTL) assays, or by assaying for T-lymphocytes specific for the antigen in a sensitized subject. Such assays are well known in the art (Erickson et al., J Immunol., (1993) 151:4189-4199; Doe et al., Eur J Immunol., (1994), 24:2369-2376). Thus, an immunological response can be one that stimulates the production of CTLs and/or the production or activation of helper T-cells. The antigen of interest can also elicit an antibody-mediated immune response that is important for the induction of protective immunity. Such assays are well described in the OIE manual (Manual of diagnostic test and vaccines for terrestrial animals, 2004 (5th edition)), Office International des Epizooties, Paris (2004), and in the literature (Tekleghiorghis et al., Clin. Vaccine Immunol., (2014) 21 (5): 674-683). Hence, an immunological response may include one or more of the following effects: the production of antibodies by B-cells and/or the activation of suppressor T-cells.

Various means for estimating or actually measuring the protective immune response generated by an FMDV mosaic vaccine preparation disclosed herein can be utilized, including without limitation, in silico analytical methods designed to determine the degree of T-cell epitope coverage provided by a particular mosaic protein or combination thereof, and in vivo methods of evaluating the FMDV mosaic vaccine preparations in animals, such as cattle.

Epitopes recognized by a T cell receptor on an FMDV-activated T cell can be identified by, for example, a ⁵¹Cr release assay or by a lymphoproliferation assay, as is well known in the art. In a ⁵¹Cr release assay, target cells that display the epitope of interest are prepared, for instance by cloning a polynucleotide encoding the epitope into an expression vector and transforming the expression vector into the target cells. Target cells are incubated with ⁵¹Cr for labeling and then mixed with subject-derived T cells, after which the cytolytic activity of T cells is measured by the release of ⁵¹Cr-bound protein into the medium.

Those skilled in the art will recognized that such analyses of efficacy are merely provided as examples. Any currently known, or later developed, assays can be chosen and utilized by the skilled artisan to determine efficacy of the vaccines detailed herein.

Having generally described this invention, the same will be better understood by reference to certain specific examples, which are included herein to further illustrate the invention and are not intended to limit the scope of the invention as defined by the claims.

### EXAMPLES

### Example 1

### Design, Construction, and In Vitro Characterization of FMDV Serotype Asia Mosaic Immunogen Cocktails

Preliminary computational analysis shows that the mosaic method of vaccine design can produce immunogens with potential to induce broadly protective immune responses, providing enhanced efficacy (protection against diverse viral isolates) and potentially result on significant cost savings (by reducing the necessity to develop, test, produce, store, and distribute separate vaccines for many different isolates). Nucleotide sequences were translated to amino-acids; the resulting protein sequences were used as input for the Epigraph algorithm (Theiler & Korber, supra), which produces output substantially equivalent to that of the original mosaic algorithm (Fischer et al, supra), but with greatly increased computational efficiency and provable optimality. A value of 9 was used for potential epitope length. Initial mosaic cocktail designs with 1, 2, 3 sequences were evaluated in terms of 9-mer amino-acid coverage. A two-sequence cocktail design was selected for vaccine construction; several different alternative “second” elements were subsequently designed, each based on a different serotype Asia subclade (see below).

For virus construction, amino-acid immunogen sequences were reverse-translated to DNA based on column-specific codon frequencies in the original nucleotide sequence alignment; each amino acid was represented by the most common nucleotide triplet that encoded that amino acid at that column in sequence alignments (not shown).

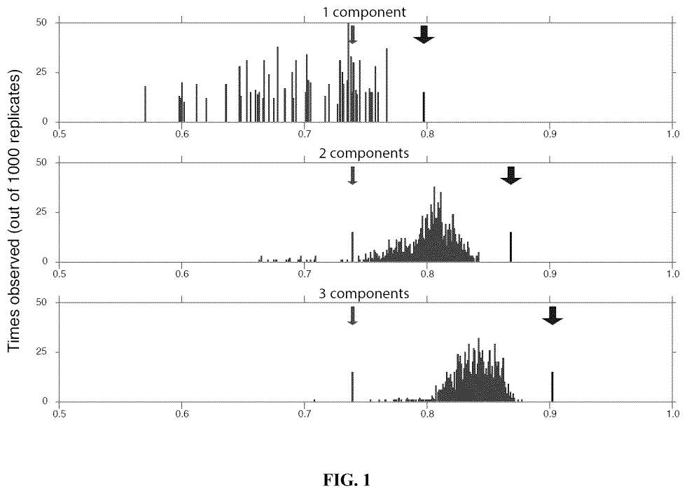

Computational scoring metrics calculated from the mosaic-FMDV capsid for FMDV serotype-Asia immunogens show marked improvement in amino-acid 9-mer coverage for 2 mosaics combined compared to monovalent and combined-natural-strain immunogens (FIG. 1)

### Construction and Derivation of Mosaic Full Genome and FMD-LL3B3D Viruses.

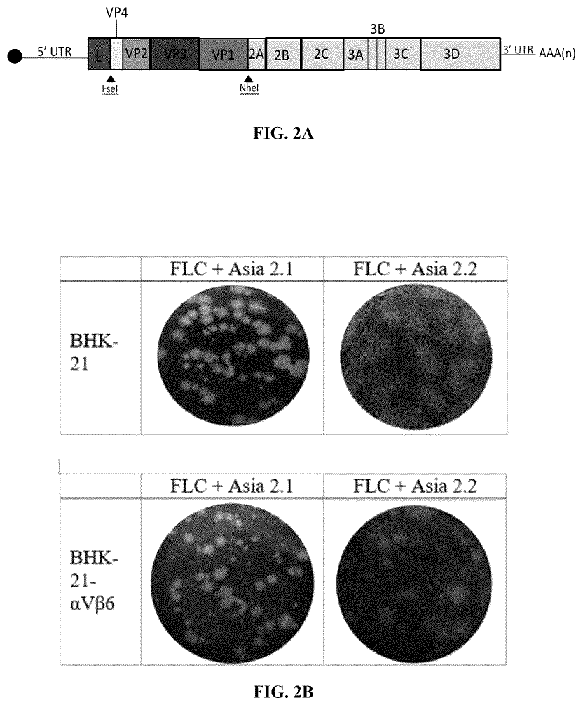

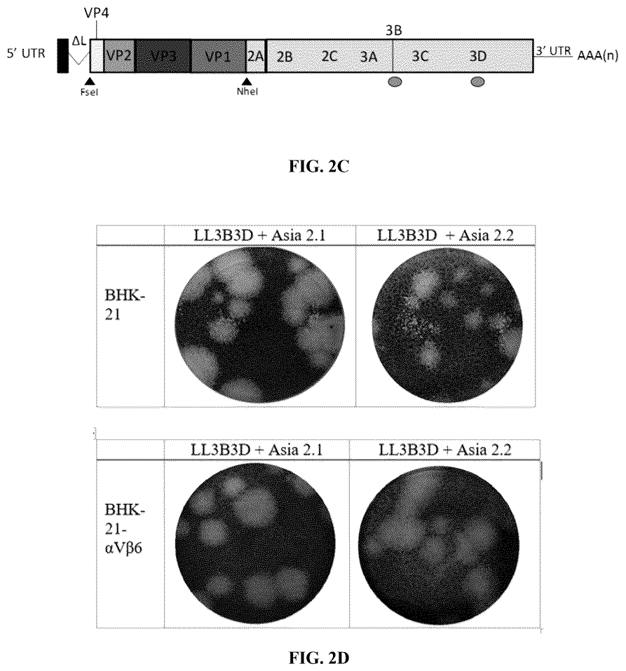

Full-length plasmids pA₂₄Cru-WT (Rieder et al, J. Virol., (2005), 20:12989-98) and pA₂₄Cru-LL3B3D (Uddowla et al, J. Virol., (2012), 86:11675-85), containing unique FseI and NheI sites in the VP4 and 2A coding regions, respectively (pA₂₄Cru-FseI/NheI) were used as templates for cloning of type Asia mosaic capsids. Capsid DNA sequences Asia Mosaic 2.1 [SEQ ID NO: 2] and 2.2 (SEQ ID NO: 4) designed using the method described by Fischer et al (Fischer, Perkins et al. 2007) were synthesized de novo (Gene Synthesis, Bio Basic Inc, Amherst, NY) and cloned into pA₂₄Cru-FseI/NheI and pA₂₄Cru-LL3B3D backbones. Specifically, 2.2 Kb FseI/NheI fragments containing P1 (capsid) mosaic sequences were substituted in pA₂₄Cru-FseI/NheI and pA₂₄Cru-LL3B3D as described in Uddowla et al., supra. cDNAs were linearized with SwaI, viral RNA was derived by in vitro transcription with T7 polymerase using a MEGAscript T7 kit (Ambion) and purified with RNeasy (Qiagen) kit following the manufacturer's directions. 5-15 μg of transcribed RNAs were electroporated into BHK-21 or BHKαVβ6 cells as previously described (Rieder et al, 2005, supra) and after a 24 h incubation at 37° C., cells were frozen for subsequent virus release and passage. Viruses were generated by passaging the virus 3-4 times in BHK-αVβ6 cells. Recovered viruses were sequenced and compared to the original plasmid DNA, and used for large scale production, in vitro characterization, and the production of inactivated vaccines. FIG. 2 shows the schematic representation of the genome and plaque morphologies of derived Asia 2.1 (SEQ ID NO: 2 (full-length); SEQ ID NO: 6 (leaderless)) and 2.2 mosaics (SEQ ID NO: 4 (full-length); SEQ ID NO: 8 (leaderless)) in both, BHK and BHKαVβ6 cell monolayers.

### Analysis of Growth of Parental and Mosaic Type Asia Viruses.

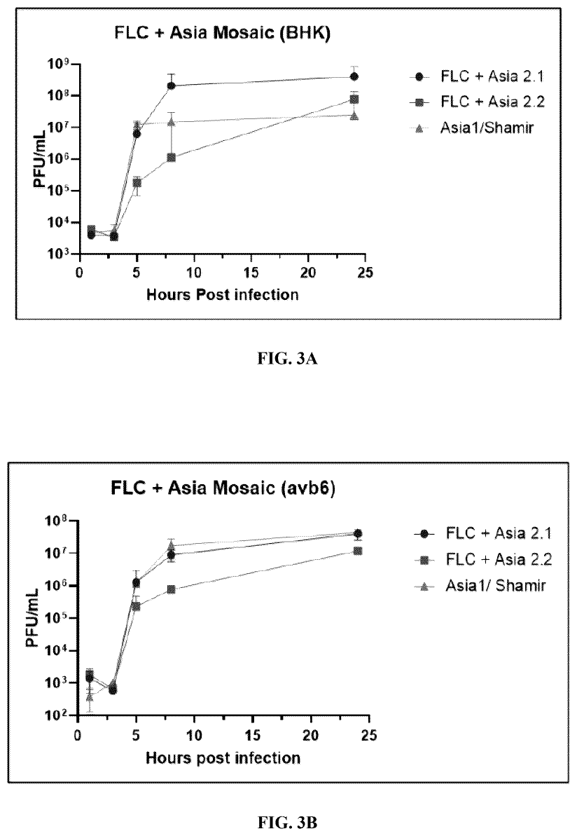

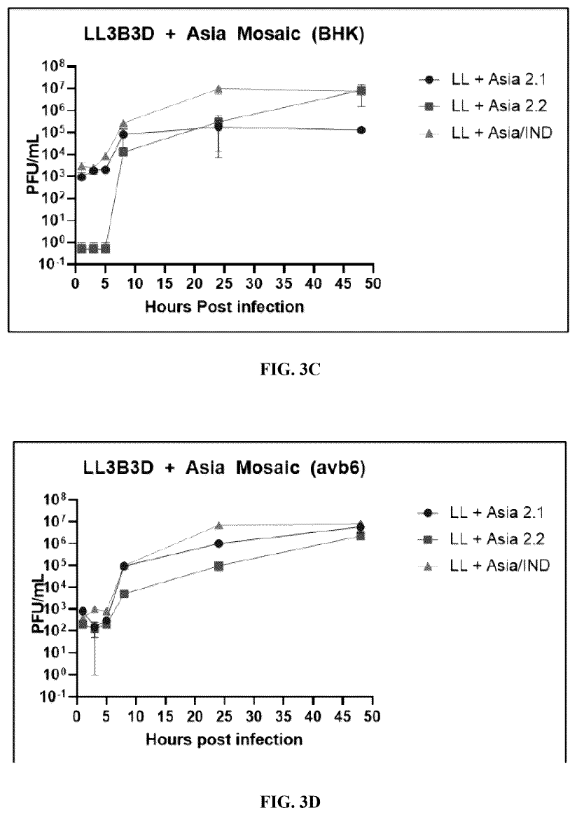

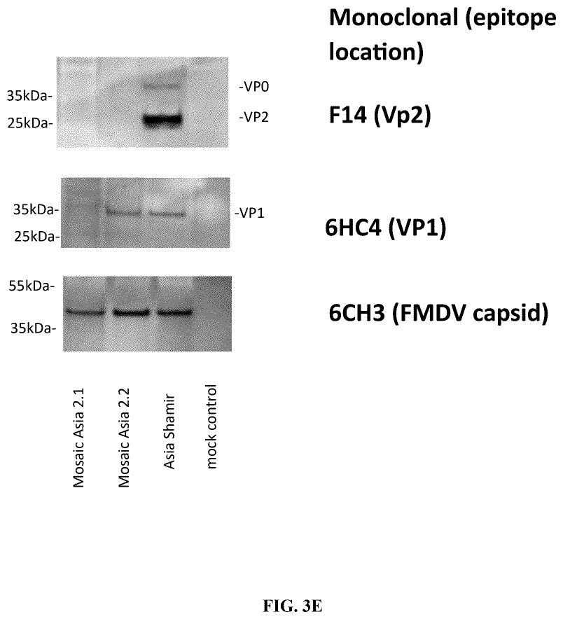

Parental (Asia IND/63/72 lineage G-Vib) and Asia Mosaic 2.1 (SEQ ID NO: 2 (full-length); SEQ ID NO: 6 (leaderless)) and 2.2 (SEQ ID NO: 4 (full-length); SEQ ID NO: 8 (leaderless)) viruses were characterized by plaque assays in BHK-21 or BHKαVβ6 cells (FIG. 3A). Plaques were visualized under a gum tragacanth overlay stained for 48 h post infection (hpi) except for FMD-LL3B3D which was stained for 72 h when titrated in BHK-21 cells. Comparative one-step growth curves between the parental Asia Shamir and Asia Mosaic 2.1 (SEQ ID NO: 2 (full-length); SEQ ID NO: 6 (leaderless)) and 2.2 (SEQ ID NO: 4 (full-length); SEQ ID NO: 8 (leaderless)) viruses were performed in BHK (FLC viruses) or BHKαVβ6 (FMD-LL3B3D viruses) cells. Pre-formed monolayers were prepared in 12-well plates and infected with the six viruses at a multiplicity of infection (MOI) of 5 at 37° C. After 1 h of adsorption at 37° C. the inoculum was removed, and the cell monolayers were rinsed with 145 mM NaCl, 25 mM morpholine-ethanesulfonic acid (MES), pH 5.5, to inactivate unabsorbed virus and then rinsed three times with BME growth media (Life Technologies, Grand Island, NY) to restore physiological pH. Plates were incubated at 37° C. in a 5% CO₂ atmosphere. At indicated times post-infection, cells were frozen, and subsequently lysed by thaw. Virus yield was measured by plaque assay on BHKαVβ6 or BHK-21 cells (Rieder et al, 2005, supra) in duplicates, as mentioned above, expressed as plaque forming units (PFU)/mL. The reactivity of Asia Mosaic and parental Asia Shamir viruses were examined using western blot analysis and using monoclonals antibodies specific to viral capsid proteins (FIG. 3B)

### Biophysical Stability of Serotype Asia Mosaic Viruses

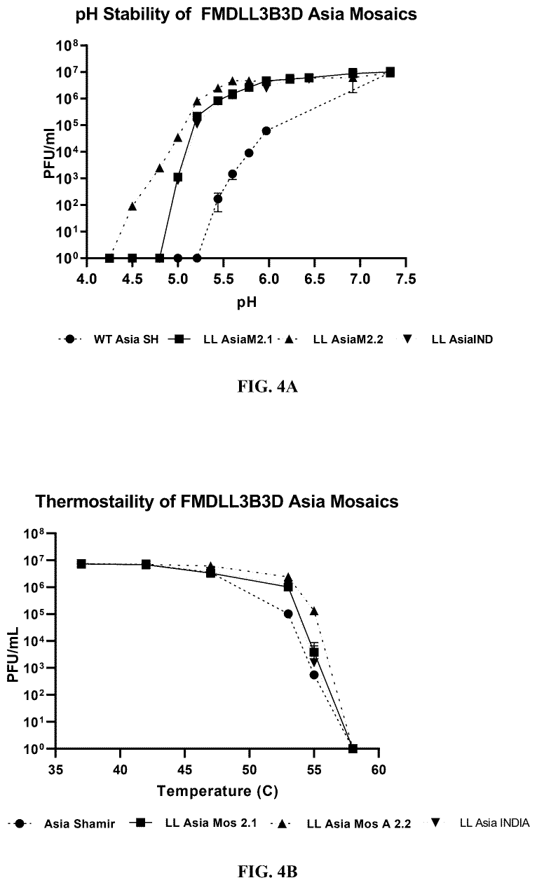

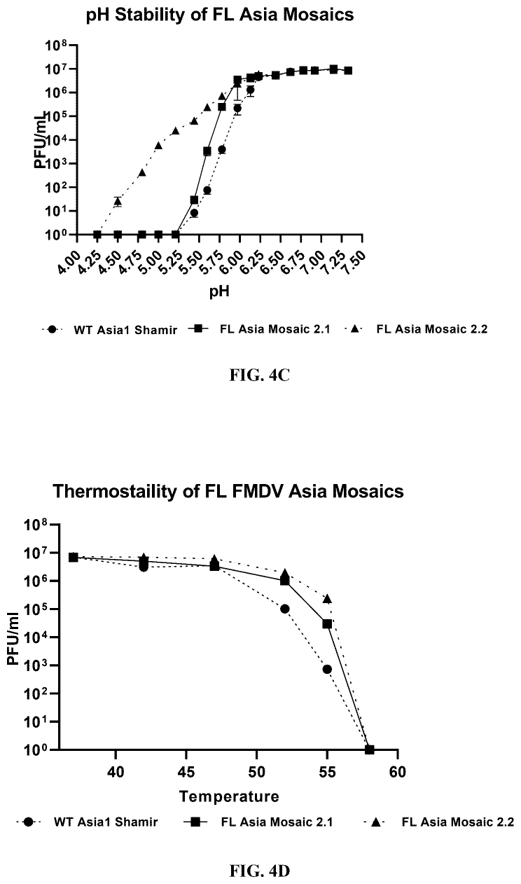

The sensitivity of FMDV to acid environment (and thus its ability to release its genome into infected cells) is an important virus particle factor to test and to further characterize the mosaic viruses, especially to compare with the wild-type viruses. A pH dissociation assay was conducted on concentrated, live viruses (FIG. 4A). The viruses were exposed to TNE buffer at pHs ranging from 7.4 to 4.25 at room temperature for 30 minutes, and then titrated onto BHKαvβ6 cells to determine viral survival. As a reference control, virus was also titrated in viral growth media (VGM) and not exposed to TNE of variable pHs. The assay was run in triplicate, on different days, and the average of the titers were calculated. The standard deviation of the triplicate runs is included in the graph (FIG. 4A). While both Asia mosaic 2.1 (SEQ ID NO: 2 (full-length); SEQ ID NO: 6 (leaderless)) and 2.2 (SEQ ID NO: 4 (full-length); SEQ ID NO: 8 (leaderless)) were more stable at a pH range from 6 to 5.5 than parental Asia1 Shamir, Asia Mosaic 2.2 required a pH of 4.25 to be completely inactivated while both Asia Mosaic 2.1 and Asia1 Shamir were completely inactivated at pH 5.25.

### Thermostability of Serotype Asia Mosaic Viruses

The sensitivity of FMDV to temperature variation was tested to further characterize the Asia Mosaic viruses as an important factor during vaccine formulation. The thermostability assay was conducted on concentrated, titrated, live viruses (FIG. 4B). The viruses were exposed to temperatures ranging from 37° C. to 58° C. while in TNE (pH 7.4), or VGM, for 30 minutes. Virus samples were then immediately placed on ice and titrated onto BHKαVβ6 cells to determine viral survival. The assay was run in triplicate, and the average of the titers were calculated. The standard deviation of the triplicate runs is included in the graph. Results indicate that both Asia Mosaics are more stable at temperatures of 52° C. and 56° C. than their parental Asia1 Shamir counterpart, while at 58° C. all three serotype Asia1 FMDV strains were completely inactivated.

### Example 2

### Vaccine Efficacy in Cattle Using Heterologous Challenge.

### Vaccine Formulation

Virus stocks were inactivated with 10 mM BEI for 24 h at 25° C. and concentrated with 8% polyethylene glycol 8000 and described (Uddowla et al., supra). The vaccines were prepared as water-in-oil-in-water (WOW) emulsion with Montadine ISA 201 (Seppic, Paris) according to the manufacturer's instructions. Briefly, the oil adjuvant was mixed into the aqueous antigen phase (50:50) at 30° C. for 15 minutes and stored at 4° C. for 24 h, followed by another brief mixing cycle for 10 minutes. The integrity of the 146S particles and antigen concentrations present in the experimental vaccines were determined by density gradient centrifugation in sucrose 10-50% (W/V) and 260 nm densitometry.

### Vaccination and Challenge of Cattle

Animal experiments were performed in the high-containment facilities at the Plum Island animal Disease Center (PIADC) in compliance with the Animal Welfare Act, the 2011 Guide for Care and Use of Laboratory Animals, the 2002 PHS policy for the human care of vertebrate animals used in testing, research and training (IRAC 1985), as well as specific animal protocols reviewed and approved by the Institutional animal care and use committee (IACUC) of PIADC (USDA.APHIS.AC certificate number 21-F-0001).

Twelve Holstein heifers (weights 250-300 kg) were divided into 4 groups as follows: one group of three animals (cow IDs R20-04, R20-05, R20-06) were immunized intramuscularly (IM) in the neck with a vaccine consisting of chemically inactivated Asia Mosaic 2.1 (SEQ ID NO: 6) and Asia Mosaic 2.2 (SEQ ID NO: 8) (4 μg+4 μg) formulated as monovalent vaccines emulsified with a commercially available water-in-oil-in-water adjuvant (Montanide ISA 201, Seppic, France). Another group of three animals (R20-10, R20-11, R20-12) were vaccinated IM with a chemically inactivated Asia Mosaic 2.1 alone (8 μg). A third group of three animals were vaccinated IM with a chemically inactivated FMD-LL3B3D Asia/India vaccine (8 μg) formulated in the same fashion (R20-13, R20-14, R20-15). One final group of three cattle (R20-01, R20-02, R20-03) were mock vaccinated IM with sterile PBS emulsified with the same adjuvant and served as the unvaccinated controls. On 21 days post-vaccination (dpv) all animals were challenged intradermolingually (IDL) with 10⁴ BTID₅₀ (50% bovine tongue infection dose) of FMDV Asia1/Shamir (Table 1).

TABLE 1
Vaccine Efficacy Study Design in cattle

| | Dose of | | |
|---|---|---|---|
| Immunization | inactivated | | Challenge |
| Group | virus | Animal ID | information |
| PBS/Control + | N/A | R22-01 | Intra-dermo |
| adjuvant ISA201 | | R22-02 | lingual with |
| | | R22-03 | FMDV-Asia |
| FMDV LL3B3D + | 4 μg Asia Mosaic | R22-04 | Shamir at 21 |
| Asia Mosaic | 2.1 + 4 μg Asia | R22-05 | DPV |
| 2.1 + 2.2 + | Mosaic 2.2 | R22-06 | |
| adjuvant ISA201 | | | |
| FMDV LL3B3D + | 8 μg Asia Mosaic | R22-10 | |
| Asia Mosaic | 2.1 | R22-11 | |
| 2.1 + adjuvant | | R22-12 | |
| ISA201 | | | |
| FMDV LL3B3D + | 8 μg LL3B3D + | R20-13 | |
| Asia/India/63/72 | Asia/India63/72 | R22-14 | |
| | | R22-15 | |

The animals were evaluated for the appearance for localized and generalized vesicular lesions at 3- and 7-days post-challenge (dpc). Clinical scores were registered as 1 credit for each affected foot and an extra credit for the presence of vesicles in the nose or mouth in addition to those that resulted from IDL inoculation for the challenge. Temperatures were collected daily, and sera and nasal secretion were collected at 1, 3, and 7 dpc. Probang cups were used to collect Oropharyngeal fluid (OPF), collected at 14, 21, and 28 dpc. OPF samples were diluted with an equal volume of minimal essential medium (MEM) containing 25 nM HEPES, cannulated for homogenization of the samples and processed for virus isolation and qRT-PCR. Aliquots intended for use in viral isolation were treated with 1,1,2-trichlorotrifluoroethane (TTE) got dissociation of the immune complexes as previous described (Brown & Cartwright, Nature, (1960), 199:1168-70) and filtered through Spin-X columns. LFBK-αVβ6 cell monolayers were inoculated with filtered TTE-treated probang cup samples. After 1 hours of adsorption, fresh DMEM media was added, and monolayers were checked daily for detection of cytopathic effect (CPE) due to the presence of FMDV. Upon detection of CPE, FMDV positivity was confirmed by rRT-PCR/sequencing on cell culture supernatants using universal FMDV primers to amplify the capsid region of the virus. Samples in which no CPE was observed were amplified through 3 blind passages to ensure there was no viable virus detected. Probang cup samples were also tested by qRT-PCR, as previously described.

### Detection of Virus in Sera and Nasal Swabs

Cattle sera and nasal secretion were assayed for the presence of virus by plaque titration on BHK-21 cells and by qRT-PCR. Viable virus titers are expressed at PFU/mL of serum or nasal secretions. The minimum detection level for this assay is 5 PFU/mL. In addition, FMDV RNA was detected by qRT-PCR as previously described (Pacheco & Mason, J. Vet. Sci., (2010), 11:133-42). Briefly, 50 μl of each sample (sera or nasal secretion) for each cow was transferred to 96-well plates (King Fisher) containing 150 μl of lysis/binding solution. RNA was then extracted using MagMax-96 viral RNA isolation kit (Ambion) on a King Fisher-96 magnetic particle processor (Thermo Electron Corp.). After an initial 5-min lysis/binding step, the RNA samples underwent a series of four washing steps, a drying step, and a final elution step. RNA was eluted in a final volume of 25 μl. At each of the above steps, RNA was magnetically bound to the beads contained in the lysis/binding solution and was transferred to the different extraction solutions. RNA extracted from all the previous described samples was analyzed in duplicate by rRT-PCR using 2.5 μl of RNA on the QuantStudio 6 with the AgPath-ID one step RT-PCR kit (applied biosystems). Cycle threshold values were converted into log 10 FMDV genome copy numbers (GCN) per mL (log 10 GCN/mL), by use of standard curves based on analysis of 10-fold dilutions of in-vitro synthesized FMDV RNA.

### Evaluation of Humoral Immune Response

Neutralizing antibody titers against FMDV Asia Mosaic vaccine strains and FMDV challenge strain Asia1-Shamir were determined by the virus neutralization test method described in the OIE Manual (OIE 2017) in 96-well plates of LFBK αVβ6 cells, on serum samples collected on days 0, 7, 14, 21, 28, 35, 42, and 49 post-vaccination. SVN titers were calculated according to the Spearman-Karber method and expressed as log 10 of the reciprocal of the final serum dilution that neutralized 100 TCID₅₀ of the respective serotype in 50% of the wells. The assay lower limit of detection was 0.45 log 10.

### Antibody Detection Against FMDV Non-Structural Proteins-Blocking ELISA

Cattle serum samples were heat-inactivated at 56° C. for 30 mins in a water bath prior to testing. The samples were then subjected to FMDV antibody detection against non-structural proteins (NSP) using a PrioCheck FMDV NS ELISA test kit (Prionics, Netherlands) following the manufacturer's instructions. A percentage inhibition (PI) of <50% is considered negative (i.e., antibodies against FMDV NSP are absent in the test sample); PI>50% is considered positive (i.e., antibodies against the FMDV NSP are present in the test sample).

### Hematology

For all animals, whole blood samples were collected in EDTA tubes and a differential cell blood counts were conducted up to 3 days post sampling (standard PIADC procedure) using a Hema Vet 950FS (Drew Scientific), according to manufacturer's instructions.

### Results and Analysis

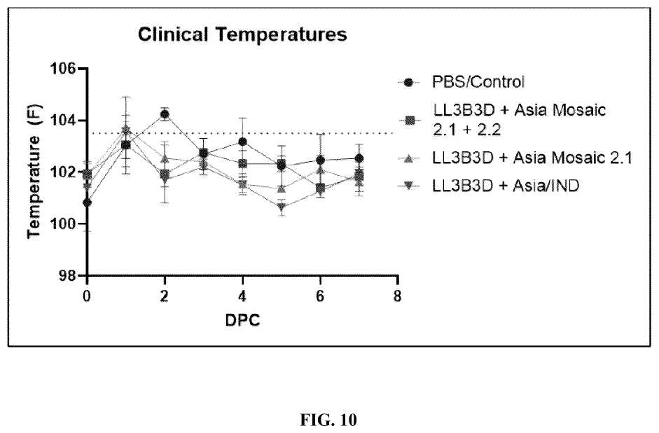

As expected, control animals developed signs of disease starting at 3 dpc, including elevated temperatures that lasted for 4 to 5 days (Table 2). Animals vaccinated with FMDV-LL3B3D Asia/India (R20-13, R20-14, R20-15) did not present with pedal or oral vesicles but did develop fever for 1-2 days. Control animals developed fever (Table 2, FIG. 10), lethargy, sialorrhea and anorexia by 1 or 2 dpc (data not shown), and observable generalized FMDV lesions by 3 dpc, pedal vesicles in all four feet and oral cavity by 7 dpc. In contrast, none of the FMDV-LL3B3D+Asia Mosaic vaccinated animals (monovalent 2.1 alone or bivalent 2.1+2.2) showed fever or clinical signs of FMDV during the experiment and all were fully protected from characteristic FMDV lesions up through 7 dpc (Table 2).

TABLE 2
Clinical scores and clinical temperatures after challenge at 21 DPV.

| | | | | | | Onset of |
|---|---|---|---|---|---|---|
| | | | | | | Fever (F.) |
| | | | | | | Peak |
| Challenge | | | DPC | | | (Duration |
| Virus | Immunization | Animal ID | 0 DPC | 3 DPC | 7 DPC | in days) |
| Intra- | PBS/Control + | R22-01 | 0 | 5 | 5 | 106.8 (5) |
| dermo | adjuvant | R22-02 | 0 | 5 | 5 | 105.4 (4) |
| lingual | ISA201 | R22-03 | 0 | 4 | 5 | 106.3 (4) |
| with | FMDV- | R22-04 | 0 | 0 | 0 | No Fever |
| FMDV- | LL3B3D + Asia | R22-05 | 0 | 0 | 0 | No Fever |
| Asia | Mosaic 2.1 + | R22-06 | 0 | 0 | 0 | No Fever |
| Shamir at | 2.2 + adjuvant | | | | | |
| 21 DPV | ISA201 | | | | | |
| | FMDV- | R22-10 | 0 | 0 | 0 | No Fever |
| | LL3B3D + Asia | R22-11 | 0 | 0 | 0 | No Fever |
| | Mosaic 2.1 + | R22-12 | 0 | 0 | 0 | No Fever |
| | adjuvant | | | | | |
| | ISA201 | | | | | |
| | FMDV- | R20-13 | 0 | 0 | 0 | 104.7 (2) |
| | LL3B3D + | R22-14 | 0 | 0 | 0 | 104 (1) |
| | Asia/India/63/72 | R22-15 | 0 | 0 | 0 | 104.2 (1) |

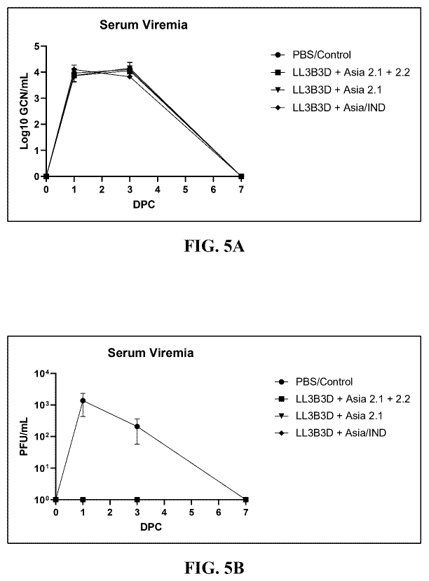

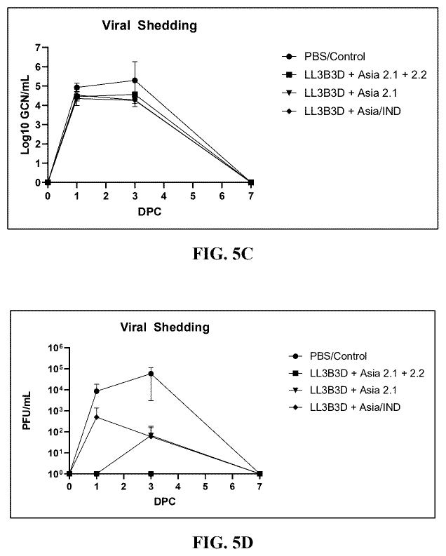

Isolation of viable virus from serum and nasal secretion was titrated on BHK-21αVβ6 monolayers stained at 48 hours post infection (FIG. 5B, FIG. 5D). Infectious virus was detected in serum samples of all the animals in control groups from 1 to 3 dpc and virus shedding was detected in nasal secretion of all control animals from the day after challenge until 3 dpc (FIG. 5B, FIG. 5D). In contrast, cattle in FMDV-LL3B3D+Asia/India group and more importantly the mono- and bi-valent FMDV-LL3B3D+Asia mosaic groups were negative for viable virus isolation from serum samples though out the experiment. Viable virus was recovered from the nasal secretion of FMDV-LL3B3D+Asia/India group at 1 and 3 dpc and from the monovalent FMDV-LL3B3D+Asia 2.1 vaccinated group at only 3 dpc. Importantly, the cattle vaccinated with the bi-valent FMDV-LL3B3D Asia Mosaic 2.1+2.2 were negative for viable virus isolation from serum samples though out the experiment, suggesting that the bi-valent FMDV-LL3B3D+Asia mosaic 2.1+2.2 provides better protection from viremia than the mono-valent FMDV-LL3B3D+Asia Mosaic 2.1 alone (FIG. 5B, FIG. 5D).

Sera and nasal secretions were also processed for RNA extraction and qRT-PCR (FIG. 5A, FIG. 5C). FMDV viral RNA was detected in control, PBS vaccinated animals challenged with FMDV Asia1/Shamir at 1 and 3 dpc in both serum and nasal swab samples. Animals vaccinated with the control parental vaccine, FMDV-LL3B3D+Asia/India, showed detectable levels of viral RNA in the nasal swab and serum samples at 1 and 3 dpc. Animals vaccinated with the bivalent FMDV-LL3B3D Asia mosaic vaccine (Asia Mosaic 2.1 (SEQ ID NO: 6)+2.2 (SEQ ID NO: 8)) showed similar levels of viral RNA present in the serum and slightly lower levels in the nasal secretion. There was a similar result observed with the animals vaccinated with the monovalent Asia 2.1 alone, in which RNA were detected in the serum and nasal secretion as well. Since no viable virus was recovered from the serum or nasal swab samples of the bi-valent FMDV-LL3B3D+Asia mosaic 2.1+2.2 vaccinated animals at any animal or at any timepoint tested, this suggests the neutralizing antibodies produced by the vaccinated animals did in fact neutralize the challenge virus and protect the animals from FMDV replication (FIG. 5A, FIG. 5B, FIG. 5C, FIG. 5D). In summary this study demonstrates that the FMDV-LL3B3D+Asia Mosaic virus adjuvanted bivalent vaccines were highly immunogenic and conferred strong protection in cattle against FMDV heterologous challenges following 21 days post vaccination.

### Analysis of Hematological Parameters

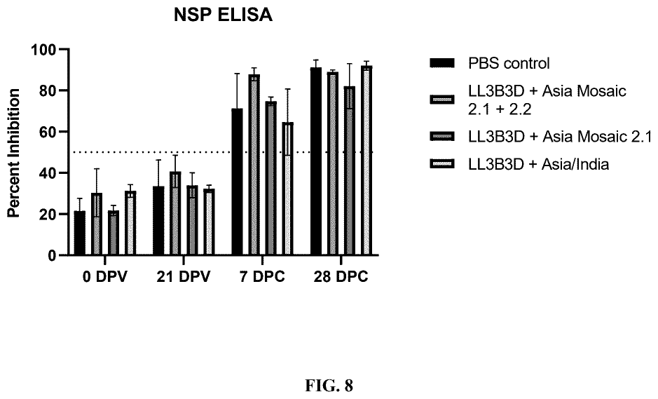

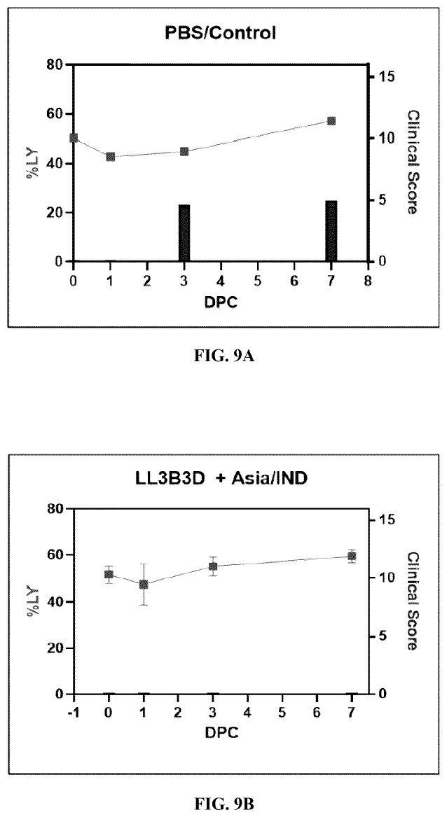

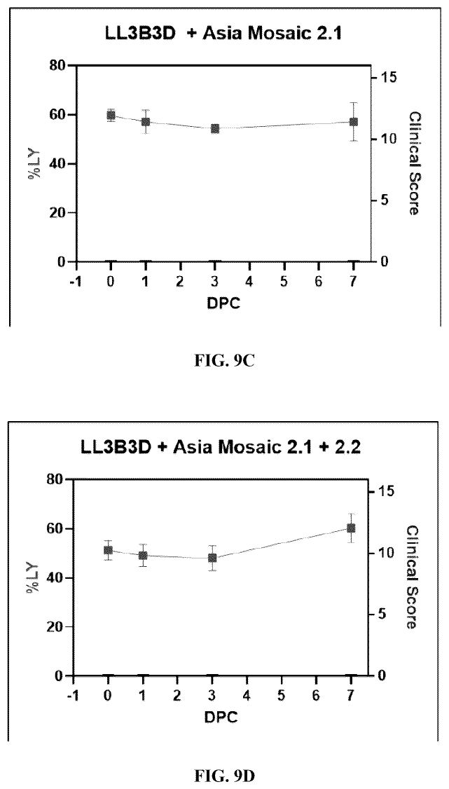

Next, the white blood cell subpopulations, specifically lymphocyte populations (FIG. 9A, FIG. 9B, FIG. 9C, and FIG. 9D) were investigated. On average, all immune cells were found with normal reference ranges parameters for cattle (data not shown).

### Humoral Responses

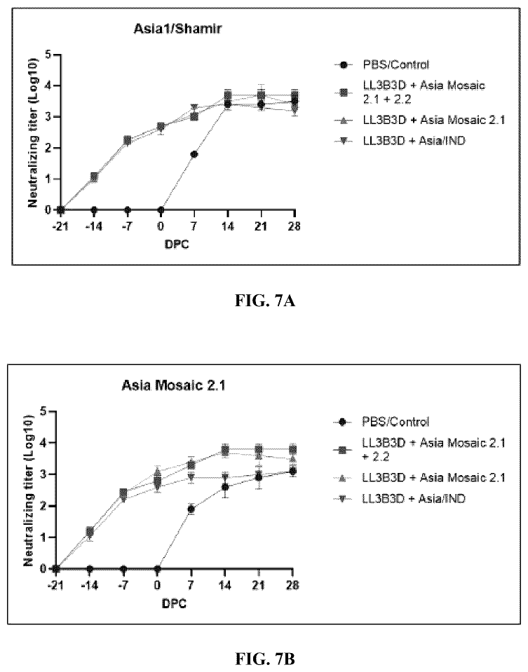

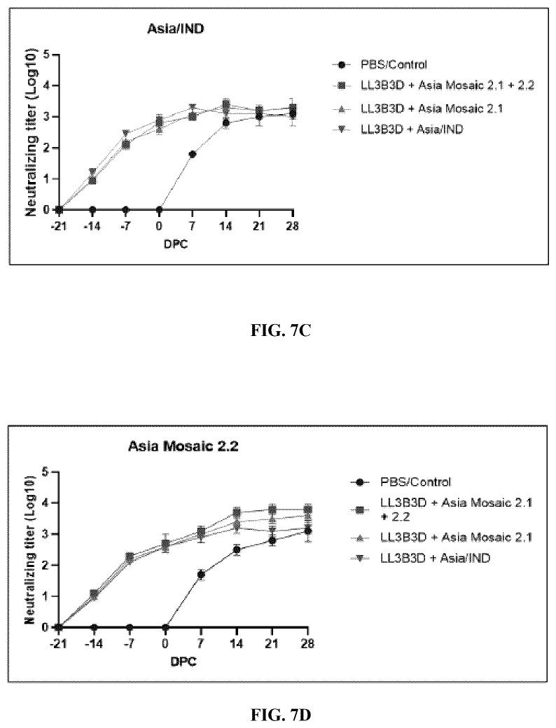

All vaccinated animals developed systemic virus neutralizing (SVN) antibodies to all four strains of FMDV serotype Asia tested by 7 dpv, reaching peak titers at the day of challenge (21 dpv), (FIG. 7A, FIG. 7B, FIG. 7C, FIG. 7D), reaching comparable titers in all vaccinated groups against challenge virus Asia1/Shamir (FIG. 7A). None of the unvaccinated control animals had SVN titers on any day prior to challenge. However, there were differences when neutralization was run against other Asia subtypes. For instance, all vaccinated animals developed serum neutralizing antibodies to Asia Mosaic 2.1 by 7 dpv, and at the day of challenge (21 dpv) neutralizing titers were higher in the animals vaccinated with both Asia Mosaic constructs or with Asia Mosaic 2.1 alone in comparison with the groups receiving the Asia/India formulations (FIG. 7B). On the other hand, all vaccinated animals developed serum neutralizing antibodies to Asia Mosaic 2.2 and Asia/India by 7 dpv, and antibody titers continued raising at 14 and 21 dpv, reaching comparable titers in all vaccinated groups at the day of challenge (21 dpv) (FIG. 7D, FIG. 7C).

### Assessment of DIVA Compatibility

Earlier studies have shown that cattle immunized with FMDV-LL3B3D marker vaccines exhibited a differential immune response compared to animals that have been infected with FMDV using a commercially available competitive 3ABC Enzyme-Linked Imunosorbent Assay (cELISA) kit, PrioCHECK FMDV NS Antibody ELISA (Thermo Fisher Scientific) following manufacturer's protocol (Uddowla et al, supra; Hardham et al, Front. Vet. Sci., (2020), 7:554305).

FIG. 8 shows Priocheck negative results for presence of antibodies against FMDV NS proteins on serum samples collected at the day of vaccination and the day of challenge (21 dpv) for all the groups. Serum samples from the vaccinated groups and PBS-mock vaccinated control groups receiving challenge with Asia1/Shamir were all positive following challenge as see at 7 dpc and 28 dpc. The results revealed that while measurable non-structural protein specific antibodies are detectable in mock and all vaccinated cattle following FMDV challenge with Asia1/Shamir virus, the levels of antibodies to FMDV were below the cut-off in animals vaccinated with the marker.

### Antigen-Specific IFNγ

Live virus for use in the ELISpot assay was grown, clarified and polyethylene glycol 8000 (PEG, Sigma) concentrated. Purified peripheral blood mononuclear cells (PBMCs) were exposed to suspensions of stimulating viruses or a positive control stimulus, PHA (Phytohemagglutinin) in the ELISpot plates and incubated at 37° C. overnight. The following day, ELISpot plates were then washed and incubated for 2 h with IFNγ detection antibody (MT307-biotin) followed by incubation with Streptavidin-HRP. Finally, these plates were washed and incubated TMB solution (Mabtech) for until clearly visible spots formed within the wells of the plate. Each ELISpot plate was immediately scanned using an ImmunoSpot plate scanner and IFNγ spots were counted using ImmunoSpot software (Cellular Technologies Ltd.). The data were normalized within animal by taking the average of the unstimulated, negative control wells and subtracting the number of spots in these wells from each treatment well of that animal before proceeding with any statistical analyses. Positive control spot counts from the PHA-stimulated wells of each animal were calculated to ensure PBMC from each animal were able to respond to ex vivo stimulation.

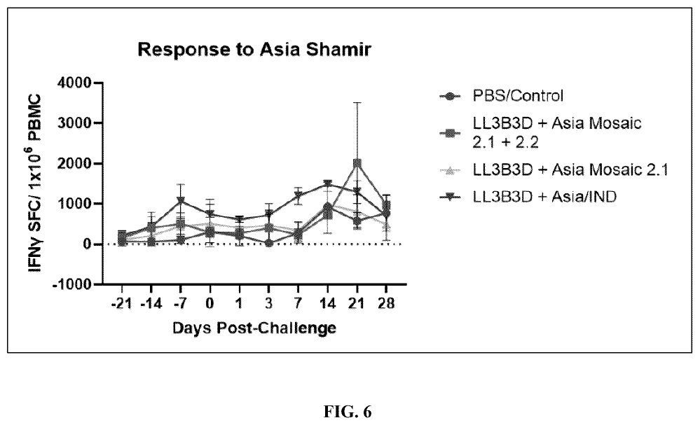

Peaks in PBMC response to Asia Shamir seen at 14 dpv in all vaccinated groups of cows with the exception of monovalent FMDV-LL3B3D+Asia Mosaic 2.1 (8 μg) vaccinated cows, which continually increased their response through 21 dpv (FIG. 6). The most profound PBMC responses to stimulation were seen in the FMDV-LL3B3D+Asia/IND 43/72 (8 μg) vaccinated group, with IFNγ spot counts in response to Asia1/Shamir (FIG. 6) rising drastically above those seen in all other groups in this study.

### Example 3

### Asia Mosaic Vaccine Efficacy Study in Swine Using Heterologous Challenge.

### Formulation of Chemically Inactivated Vaccines

Chemically inactivated FMDV-LL3B3D Asia Mosaic viruses (Mosaics 2.1 and 2.2) were formulated using Seppic ISA 201. A parental FMDV-LL3B3D Asia/India virus produced in the same FMDV-LL3B3D backbone was inactivated and formulated with the same adjuvant for this vaccine efficacy study in swine. The study design consists of bivalent formulations with and without a boost at 14 dpv are shown in (Table 3).

### Asia Mosaic Vaccine Efficacy Study in Swine Using Heterologous Challenge.

Sixteen Yorkshire gilts (five weeks old and weighing approximately 18-23 kg each) were acclimated for 1 week and were subsequently divided into 4 groups of 4 animals each (Table 3). In group 1, the animals each received 4 μg of chemically inactivated FMDV-LL3B3D Asia 2.1+2.2 and 7.5 μg of FMDV-LL3B3D O mosaic 2.1 and 2.2.7 (quadrivalent vaccine) formulated with Seppic ISA 201 adjuvant. O mosaic 2.1 and 2.2.7 are described fully in U.S. patent application Ser. No. 17/889,737, which is incorporated by reference. In group 2, the animals received 4 μg of chemically inactivated FMDV-LL3B3D Asia 2.1+2.2 (bivalent). Animals in group 3, received 8 μg of chemically inactivated parental FMDV-LL3B3D+Asia/India. Finally, animals in group 4 were mock vaccinated with PBS formulated with the adjuvant. Animals in group 1 (quadrivalent) were boosted at 14 dpv. At 28 dpv, all animals were challenged with FMDV Asia1/Shamir inoculated intradermally in the heel bulb (IDHB) of the right hind foot with 10⁴ TCID₅₀/animal. The animals were evaluated for the appearance of localized and generalized lesions at 1-, 2-, 3-, 5-, and 7-dpc. Clinical scores were registered for each affected foot outside the challenge site, and for the presence of vesicles in the nose or mouth. Temperatures were collected daily and sera and nasal swabs were collected at 1-, 3-, and 7-dpc.

TABLE 3
Vaccine Efficacy Study Design in swine

| Immunization | Dose of Inactivated | | | Challenge |
|---|---|---|---|---|
| Group | Vaccine | Boost | Animal ID | information |
| FMDV- | 4 μg each of Asia | Yes | 38 | FMDV |
| LL3B3D + | 2.1 + Asia 2.2 | Boost | 39 | Asia1/Shamir |
| Asia Mosaic | 7.5 μg each of O | at 14 | 40 | (Heel Bulb |
| 2.1 + 2.2 + | 2.1 + O 2.2.7 | DPV | | Intradermal) |
| O Mosaic | | | | |
| 2.1 + 2.2.7 | | | | |
| FMDV- | 4 μg each of Asia | No | 42 | |
| LL3B3D + | 2.1 + Asia 2.2 | | 43 | |
| Asia Mosaic | | | 44 | |
| 2.1 + 2.2 | | | 45 | |
| FMDV- | 8 μg of Asia/India | No | 46 | |
| LL3B3D + | | | 47 | |
| Asia/India | | | 48 | |
| | | | 49 | |
| PBS/Control | N/A | No | 50 | |
| | | | 51 | |
| | | | 52 | |

### Clinical Outcomes

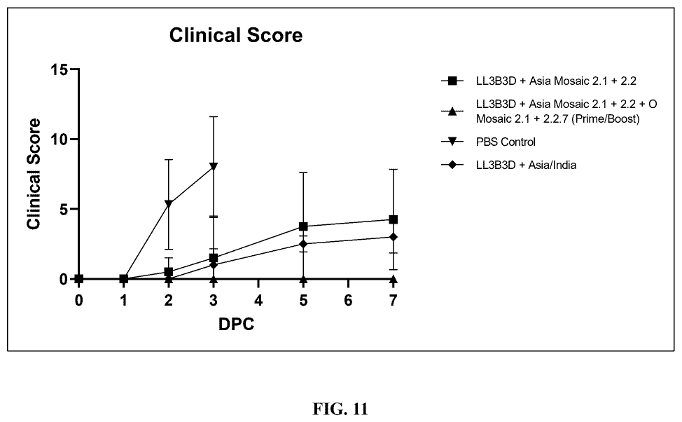

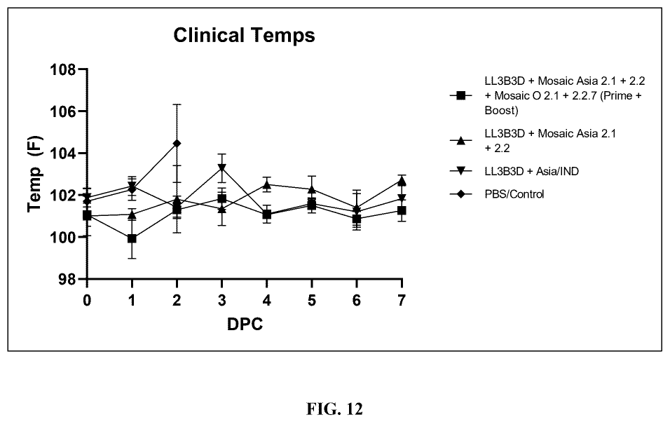

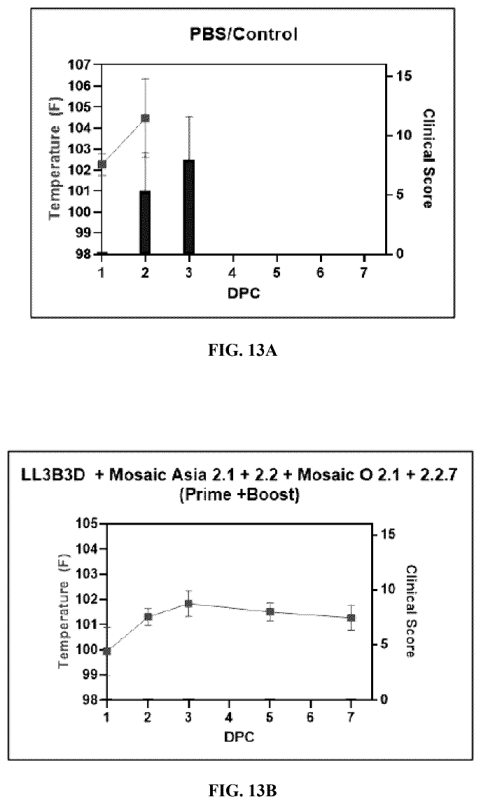

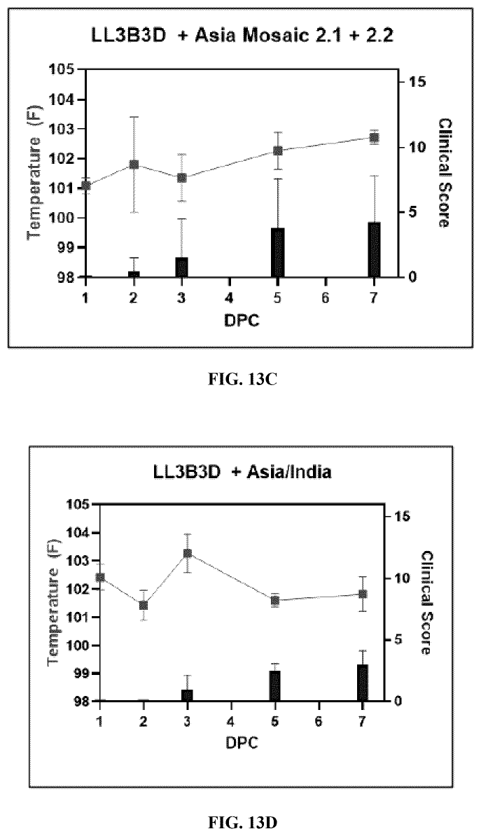

For the data shown in Table 4, animals were assessed for clinical temperatures daily and FMDV lesions at 1, 2, 3, 5 and 7 dpc. Importantly, animals vaccinated with quadrivalent FMDV-LL3B3D+Asia 2.1+2.2+O mosaic 2.1 and 2.2.7 (prime+boost) showed no clinical signs though out the course of the experiment and therefor for protected from clinical FMDV heterologous challenge. Additionally, these animals never demonstrated a fever after challenge with hyper virulence FMDV Asia1/Shamir (Table 4, FIG. 11, FIG. 12, FIG. 13A, FIG. 13B, FIG. 13C, and FIG. 13D). These data are also significant as they suggest that there is no interference when boosted animals are vaccinated simultaneously with an FMDV-LL3B3D O mosaic vaccine. All animals vaccinated with bivalent FMDV-LL3B3D+Asia Mosaic 2.1+2.2, showed clinical signs starting at 3 dpc, suggesting that a prime+boost strategy is more efficacious than 1 single vaccination. Similarly, animals vaccinated with LL3B3D+Asia/India showed clinical signs and 2/4 animals showed fever (Table 4, FIG. 11, FIG. 12, FIG. 13A, FIG. 13B, FIG. 13C, and FIG. 13D). Control animals developed fever (Table 4, FIG. 11, FIG. 12, FIG. 13A, FIG. 13B, FIG. 13C, and FIG. 13D) and observable generalized FMDV lesions by 3 dpc. All control animals had to be euthanized by 3 dpc, suggesting that this was a hyper virulent strain of FMDV Asia1/Shamir.

TABLE 4
Clinical scores and temperatures in vaccinated and
control animals challenged with FMDV Asia/Shamir

| Immunization | Animal | DPC | | | | | Onset of Fever (° F.) |
|---|---|---|---|---|---|---|---|
| Group | ID | 1 | 2 | 3 | 5 | 7 | (Duration in days) |
| LL3B3D + Mosaic Asia | #38 | 0 | 0 | 0 | 0 | 0 | No |
| 2.1 + 2.2 Mosaic O2.1 + | #39 | 0 | 0 | 0 | 0 | 0 | No |
| 2.2.7 | | | | | | | |
| Prime + Boost | #40 | 0 | 0 | 0 | 0 | 0 | No |
| LL3B3D + Mosaic Asia | #42 | 0 | 0 | 0 | 0 | 1 | No |
| 2.1 + 2.2 | #43 | 0 | 2 | 6 | 9 | 9 | 103.9 (1) |
| | #44 | 0 | 0 | 0 | 2 | 2 | No |
| | #45 | 0 | 0 | 0 | 4 | 5 | No |
| LL3B3D + Asia/India | #46 | 0 | 0 | 2 | 3 | 4 | 103.9 (1) |
| | #47 | 0 | 0 | 2 | 3 | 4 | 103.8 (1) |
| | #48 | 0 | 0 | 0 | 2 | 2 | No |
| | #49 | 0 | 0 | 0 | 2 | 2 | No |
| PBS Control | #50 | 0 | 3 | 7 | ND | ND | ND |
| | #51 | 0 | 9 | 12 | ND | ND | 106.2 (ND) |
| | #52 | 0 | 4 | 5 | ND | ND | 104.7 (ND) |

### Detection of Virus in Pig Serum and Nasal Swabs

Pig serum and nasal swab samples were assessed for the presence of viable virus at 1, 3, and 7-dpc by plaque assay on BHK-21 αVβ6 monolayers under a tragacanth overlay. Virus titers were expressed as (+) if visible plaques were observed or (−) if no plaques were visible; ND indicates that animals were euthanized before the sample was collected (Table 5). Data provided showed no live, replicating viremia in the serum samples from any vaccinated animal though out the duration of the experiment (Table 5). In contrast, live replicating virus was isolated from serum of PBS vaccinated control animals though out the duration of the experiment (Table 5).

TABLE 5
Viral isolation in serum samples from vaccinated and
control animals challenged with FMDV Asia/Shamir.

| | | 1 DPC | 3 DPC | 7 DPC |
|---|---|---|---|---|
| | Animal | Viral | Viral | Viral |
| Immunization Group | ID | Isolation | Isolation | Isolation |
| FMDV-LL3B3D + | #38 | − | − | − |
| Mosaic Asia 2.1 + 2.2 | #39 | − | − | − |
| Mosaic O2.1 + 2.2.7 | #40 | − | − | − |
| Prime + Boost | | | | |
| FMDV-LL3B3D + | #42 | − | − | − |
| Mosaic Asia 2.1 + 2.2 | #43 | − | − | ND |
| | #44 | − | − | − |
| | #45 | − | − | − |
| FMDV-LL3B3D + | #46 | − | − | ND |
| Asia/India | #47 | − | − | − |
| | #48 | − | − | − |
| | #49 | − | − | − |
| PBS Control | #50 | + | ND | ND |
| | #51 | + | − | ND |
| | #52 | + | + | ND |

### Detection of Virus in Pig Serum and Nasal Swabs

Data provided showed no live, replicating viral shedding in the nasal swab samples from any quadrivalent vaccinated animal though out the duration of the experiment (Table 6). Only 1 animal (#43) showed viable virus recovered from a nasal swab sample 3 dpc in the bi-valent (Asia 2.1 (SEQ ID NO: 6)+2.2 (SEQ ID NO: 6)) vaccinated group. In contrast, live replicating virus was isolated from nasal swab samples of PBS vaccinated control animals though out the duration of the experiment (Table 6).

TABLE 6
Viral isolation in nasal swab samples from vaccinated
and control animals challenged with FMDV Asia/Shamir.

| | | 1 DPC | 3 DPC | 7 DPC |
|---|---|---|---|---|
| | Animal | Viral | Viral | Viral |
| Immunization Group | ID | Isolation¹ | Isolation | Isolation |
| FMDV-LL3B3D + | #38 | − | − | − |
| Mosaic Asia 2.1 + 2.2 | #39 | − | − | − |
| Mosaic O2.1 + 2.2.7 | #40 | − | − | − |
| Prime + Boost | | | | |
| FMDV-LL3B3D + | #42 | − | − | − |
| Mosaic Asia 2.1 + 2.2 | #43 | − | + | ND |
| | #44 | − | − | − |
| | #45 | − | − | − |
| FMDV-LL3B3D + | #46 | − | − | ND |
| Asia/India | #47 | − | − | − |
| | #48 | − | − | − |
| | #49 | − | − | − |
| PBS Control | #50 | − | ND | ND |
| | #51 | + | + | ND |
| | #52 | − | + | ND |

### Analysis of Hematological Parameters

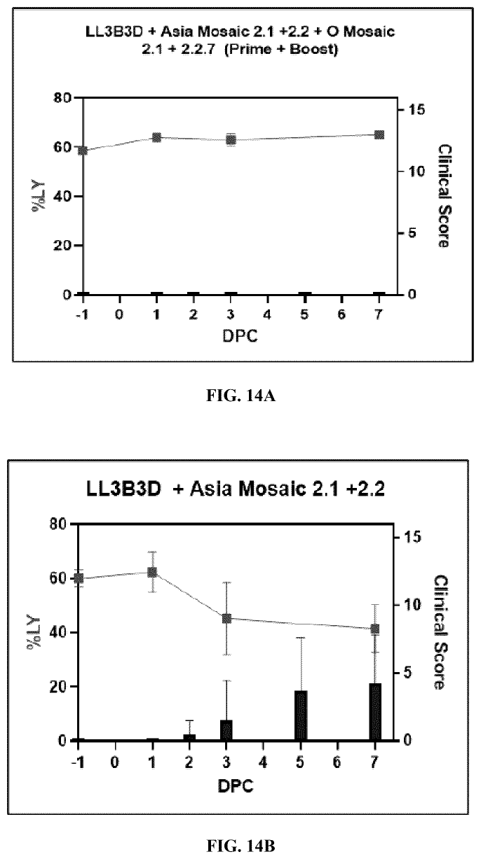

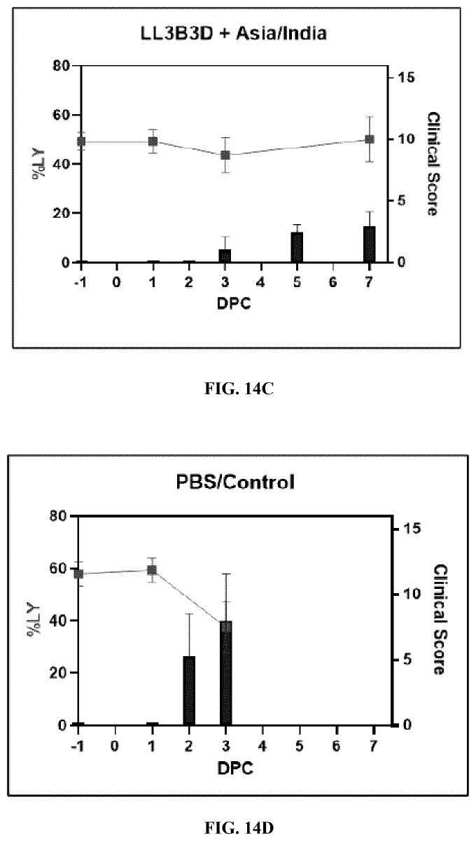

White blood cell subpopulations, specifically lymphocyte populations (FIG. 14A, FIG. 14B, FIG. 14C, and FIG. 14D) were investigated. On average, all immune cells were found with normal reference ranges parameters for cattle (data not shown). Lymphocyte population decreased dramatically in PBS vaccinated animals challenged with Asia1/Shamir which correlated with a rise in clinical scores in the animals. A similar pattern was observed in those animals vaccinated with bivalent FMDV-LL3B3D+Asia Mosaic 2.1 (SEQ ID NO: 6)+2.2 (SEQ ID NO: 8). In contrast, the animals vaccinated with the quadrivalent FMDV-LL3B3D Asia Mosaic 2.1+2.2+O mosaic 2.1+2.2.7 did not show any noticeable decrease in lymphocyte population, which correlated with the lack of clinical lesion observed in the animals (FIG. 14A, FIG. 14B, FIG. 14C, and FIG. 14D).

### Assessment of DIVA Compatibility

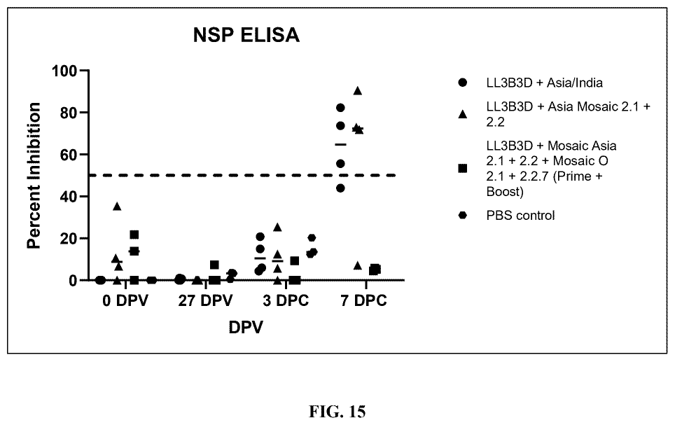

FIG. 15 shows Priocheck negative results for presence of antibodies against FMDV NS proteins on serum samples collected at the day of vaccination and the day of challenge (28 dpv) for all the groups. Serum samples from the PBS-mock vaccinated control groups receiving challenge with Asia1/Shamir were all positive following challenge as see at 7 dpc. Similar results were observed for the animals vaccinated with bivalent FMDV-LL3B3D+Asia Mosaic Asia Mosaic 2.1 (SEQ ID NO: 6)+2.2 (SEQ ID NO: 8). The results revealed that while measurable non-structural protein specific antibodies are detectable in mock and all vaccinated swine following FMDV challenge with Asia1/Shamir virus, the levels of antibodies to FMDV were below the cut-off in animals vaccinated with the marker. Negative values for antibodies against FMDV NS proteins at before challenge was consistent with the expected DIVA capability of the FMDV-LL3B3D vaccine platform.

In contrast, animals vaccinated with the quadrivalent FMDV-LL3B3D Asia Mosaic Asia Mosaic 2.1 (SEQ ID NO: 6)+2.2 (SEQ ID NO: 8)+O mosaic 2.1+2.2.7 did not show presence of antibodies against FMDV NS proteins at any point during the experiment. This suggests that these animals were protected from virulent FMDV challenge and there was likely no viable virus or sero-conversion (FIG. 15).

### T-Cells IFNγ ELIspot Results on Asia Mosaic Vaccines in Swine Vaccine Efficacy Study

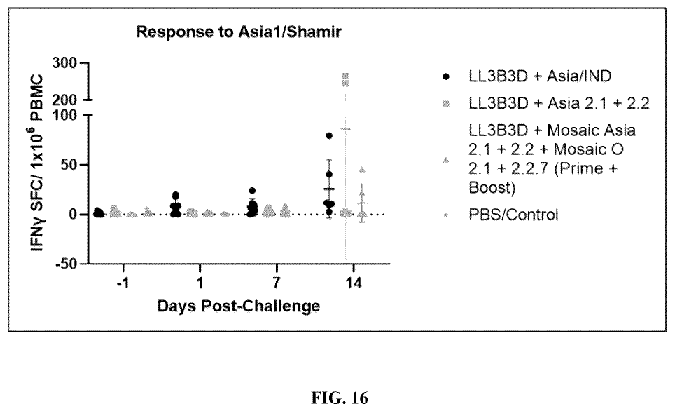

At each sampling time point, heparinized blood was collected from each pig. PBMCs were isolated from the whole blood, stimulated with live virus, and assayed for T-cell IFNγ production, as described above. In all vaccinated animals, T-cell IFNγ responses increased over time after challenge in response to FMDV Asia1/Shamir stimulation. This was not observed in PBS vaccinated animals (FIG. 16).

### Serum Virus Neutralization

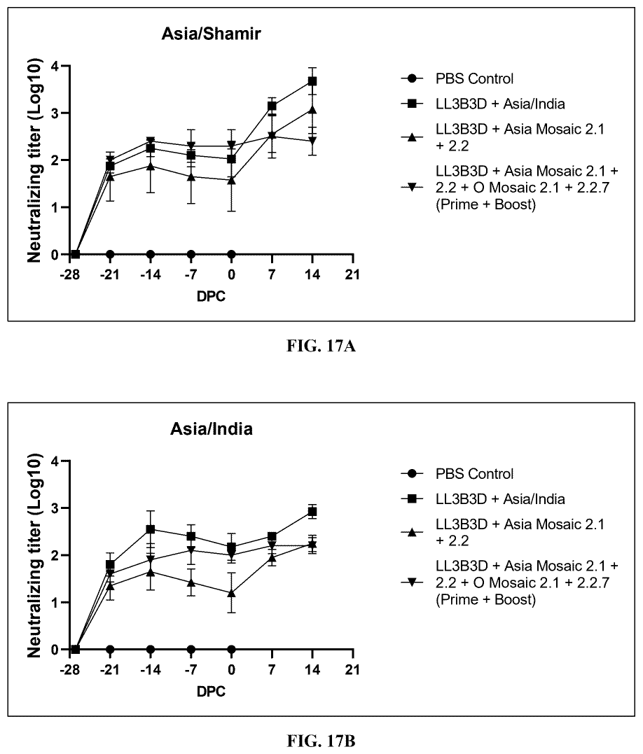

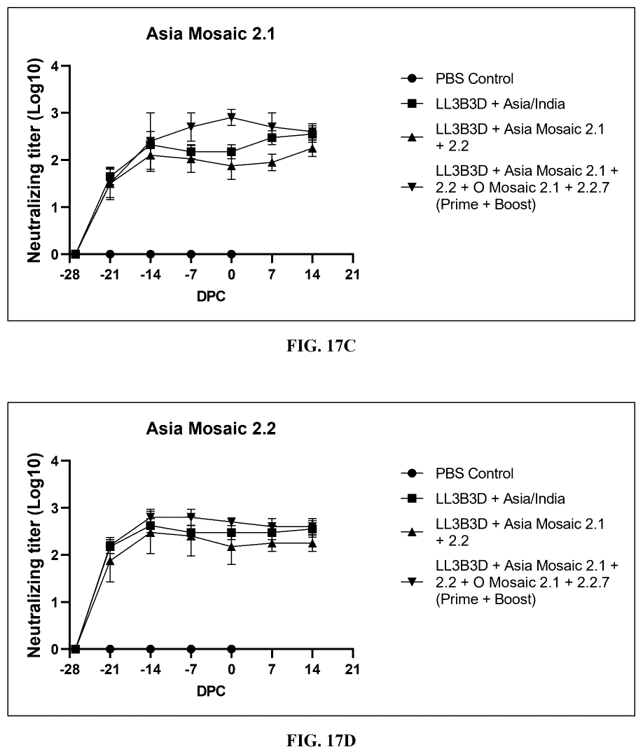

All animals vaccinated with FMDV Asia Mosaics or Asia India developed virus neutralizing antibodies against all four strains of FMDV Asia tested, starting at 7 dpv, reaching a peak at 14-21 dpv and persisting until the day of the challenge at 28 dpv (FIG. 17A, FIG. 17B, FIG. 17C, and FIG. 17D). None of the unvaccinated control animals had SVN titers on any sampling day prior to challenge against any of the four strains of FMDV Asia tested (FIG. 17A, FIG. 17B, FIG. 17C, and FIG. 17D). After challenge, all the animals in the group immunized twice with FMDV serotypes Asia and O mosaic constructs were completely protected from generalized clinical FMD and there was no significant increase in neutralizing titers against any of the four FMDV Asia strains tested. On the contrary, the animals in the groups that were immunized with a single dose of either Asia Mosaics or Asia India, even when there was a delay in the presentation and a reduction in the severity of the disease, three out of four animals in the first group and all the animals in the second group developed FMD clinical signs. In both experimental groups, the animals showed an increase in neutralizing antibody titers against Challenge FMDV Asia Shamir, indicating a humoral immune response after challenge exposure. All the pigs in the unvaccinated control group had to be euthanized by 3 dpc due to the severity of FMDV clinical signs in accordance with IACUC regulations and as a result no samples were available from this group to test for neutralizing antibody titers after challenge inoculation.

### Example 4

### Multivalent Mosaic (A, O, Asia) Efficacy Study in Cattle

### Animal Study Design

Animal experiments were performed in the high-containment facilities of the Plum Island Animal Disease Center (PIADC), in compliance with the Animal Welfare Act (AWA), the 2011 Guide for Care and Use of Laboratory Animals, the 2002 PHS Policy for the Humane Care and Use of Laboratory Animals, and U.S. Government Principles for Utilization and Care of Vertebrate Animals Used in Testing, Research and Training (IRAC 1985), as well as specific animal protocols reviewed and approved by the Institutional Animal Care and Use Committee (IACUC) of PIADC (USDA/APHIS/AC certificate number 21-F-0001).

Eighteen Holstein heifers, between 250 and 300 kg were allowed to acclimate from shipping for 1 week before the start of the study (Table 7). Four groups of three animal each were immunized with a trivalent experimental inactivated vaccine that included FMDV Asia Mosaics 2.1 (8 μg of inactivated virus antigen), FMDV O Mosaics 2.1 (20 μg of inactivated virus antigen) and FMDV A Mosaics 2.2 (10 μg of inactivated virus antigen) formulated as an emulsion with a commercially available water-in-oil-in-water adjuvant (Montanide ISA 201) and was administered by intramuscular inoculation in the neck. Two of those groups received a boost immunization of the same trivalent formulation 14 days after the prime inoculation. The remaining two groups were mock vaccinated with sterile PBS emulsified with Montanide ISA 201 adjuvant and served as unvaccinated controls. On day 28 post prime vaccination (dpv), three groups (one group of single vaccinated, another group of prime and boost vaccinated and a mock vaccinated control group) were challenged intradermolingually (IDL) with 10⁴ BTID₅₀ (50% bovine tongue infectious doses) of FMDV A Iran 05. The remaining three groups of cattle were challenged with FMDV O1 Manisa using the same dose and route of inoculation.

TABLE 7
Trivalent Mosaic Vaccine efficacy study design in cattle.

| Immunization | | Dose of | Animal | Challenge |
|---|---|---|---|---|
| Group | Boost | inactivated virus | ID | information |
| Trivalent (A + | Yes | A Mosaic 2.2 (10 ug) | R23-35 | FMDV |
| O + Asia) + | | O Mosaic 2.1 (20 ug) | R23-36 | O1 Manisa |
| adjuvant | | Asia Mosaic 2.1 (8 ug) | R23-37 | (10⁴ BTID₅₀) |
| ISA201 | | | | |
| Trivalent (A + | | A Mosaic 2.2 (10 ug) | R23-38 | |
| O + Asia) + | No | O Mosaic 2.1 (20 ug) | R23-39 | |
| adjuvant | | Asia Mosaic 2.1 (8 ug) | R23-40 | |
| ISA201 | | | | |
| PBS + | N/A | N/A | R23-41 | |
| adjuvant | | | R23-42 | |
| ISA201 | | | R23-43 | |
| Trivalent (A + | Yes | A Mosaic 2.2 (10 ug) | R23-44 | FMDV |
| O + Asia) + | | O Mosaic 2.1 (20 ug) | R23-45 | A Iran 05 |
| adjuvant | | Asia Mosaic 2.1 (8 μg) | R23-46 | (10⁴ BTID₅₀) |
| ISA201 | | | | |
| Trivalent (A + | No | A Mosaic 2.2 (10 ug) | R23-47 | |
| O + Asia) + | | O Mosaic 2.1 (20 ug) | R23-48 | |
| adjuvant | | Asia Mosaic 2.1 (8 ug) | R23-49 | |
| ISA201 | N/A | N/A | R23-50 | |
| PBS + | | | R23-51 | |
| adjuvant | | | R23-52 | |
| ISA201 | | | | |

The animals were then monitored at 3- and 7-days post-challenge (dpc) for the appearance of localized and generalized clinical lesions. Also, at 1, 3 and 7 dpc nasal swabs (cotton tip, immersed in 2 mL of minimum essential medium with 25 mM HEPES), sera, and heparinized blood were collected, and body temperatures were recorded daily. Clinical signs were scored as 1 credit for each affected foot and an extra point for the presence of vesicles in the mouth that were not related to the lingual sites of challenge virus inoculation.

### Clinical Results

All animals in the groups immunized with trivalent (A, O, Asia) Mosaic vaccine were protected from generalized clinical FMD after intradermolingual challenge with either FMDV A Iran 05 or FMDV 01 Manisa (Table 7) at 10⁴ BTID₅₀/animal. As expected, all mock vaccinated control animals showed generalized lesions of FMD and fever between 3 and 5 days after challenge. Animals were assessed for clinical temperatures daily after challenge with either FMDV O1/Manisa or A/Iran/05 (Table 8). One out of three animals vaccinated with the FMDV trivalent mosaic vaccine (single dose) and challenged with FMDV 01/Manisa did develop fever, approximately 24 hours after challenge lasting for only 1 day. However, animals that received this trivalent vaccine in addition to a booster at 21 days post-vaccination did not develop fever at any point following challenge with virulent FMDV 01/Manisa. As expected, control vaccinated animals challenged with FMDV O1/Manisa developed fever 24 hours after challenge, lasting for 2-4 days.

TABLE 8
Clinical scores and clinical temperatures after challenge.

| | | | | | | Onset of |
|---|---|---|---|---|---|---|
| | | | | | | Fever Peak |
| Challenge | | Animal | DPC | | | (Duration in |
| Virus | Immunization Group | ID | 0 DPC | 3 DPC | 7 DPC | days) |
| FMDV | Trivalent (A + O + Asia) + | R23-35 | 0 | 0 | 0 | No Fever |
| O1 Manisa | adjuvant ISA201 + Boost | R23-36 | 0 | 0 | 0 | No Fever |
| (10⁴ BID₅₀) | | R23-37 | 0 | 0 | 0 | No Fever |
| | Trivalent (A + O + Asia) + | R23-38 | 0 | 0 | 0 | No Fever |
| | adjuvant ISA201 | R23-39 | 0 | 0 | 0 | No Fever |
| | | R23-40 | 0 | 0 | 0 | 106.4 (1) |
| | PBS + adjuvant ISA201 | R23-41 | 0 | 5 | 5 | 104.9 (2) |
| | | R23-42 | 0 | 5 | 5 | 105.2 (2) |
| | | R23-43 | 0 | 4 | 5 | 105.1 (2) |
| FMDV | Trivalent (A + O + Asia) + | R23-44 | 0 | 0 | 0 | No Fever |
| A Iran 05 | adjuvant ISA201 + Boost | R23-45 | 0 | 0 | 0 | No Fever |
| (10⁴ BID₅₀) | | R23-46 | 0 | 0 | 0 | No Fever |
| | Trivalent (A + O + Asia) + | R23-47 | 0 | 0 | 0 | 103.9 (1) |
| | adjuvant ISA201 | R23-48 | 0 | 0 | 0 | 104.9 (2) |
| | | R23-49 | 0 | 0 | 0 | 104.9 (2) |
| | PBS + adjuvant ISA201 | R23-50 | 0 | 5 | 5 | 104.9 (2) |
| | | R23-51 | 0 | 2 | 3 | 106.5 (4) |
| | | R23-52 | 0 | 2 | 4 | 104.2 (2) |

Animals vaccinated with the FMDV trivalent mosaic vaccine (single dose) and challenged with FMDV A/Iran/05 did develop fever by 24 hours post-challenge lasting for approximately 2 days. However, two animals that received this trivalent vaccine in addition to a booster at 21 days post-vaccination only developed a mild fever immediately following challenged, which resolved quickly post-challenge. As expected, control vaccinated animals challenged with FMDV A/Iran/05 developed fever 24 hours after challenge, lasting for 4 days before resolving.

### Serum Viremia (Detection of Infectious Virus)

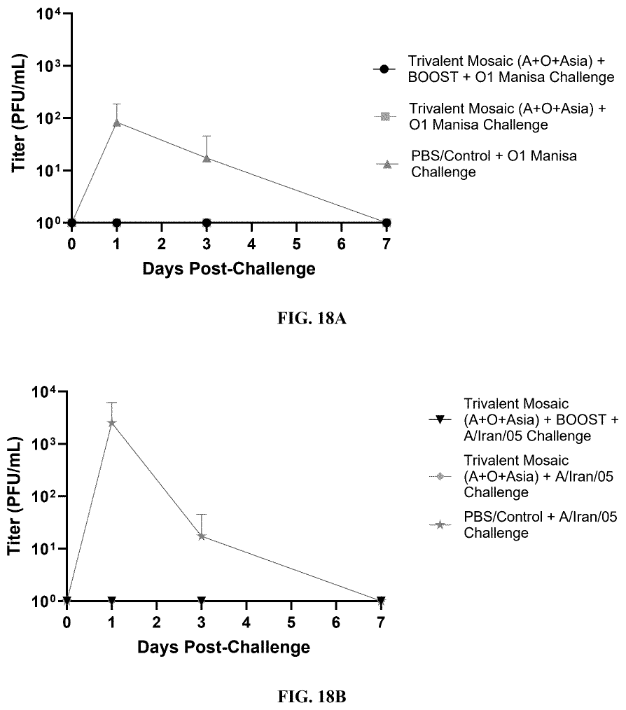

Isolation of viable virus from serum was titrated on BHK-21 αVβ6 monolayers under a tragacanth overlay and stained with crystal violet at 24 hours post infection (FIG. 18A and FIG. 18B). FMDV O1/Manisa challenge virus was isolated from serum samples of all the animals in control groups from 1 to 3 days post-challenge. In contrast, cattle that received the FMDV trivalent mosaic vaccine (both single dose and boosted) were negative for viable virus isolation from serum samples throughout the duration of the experiment (FIG. 18A). Likewise, FMDV A/Iran/05 challenge virus was isolated from serum samples of all the animals in control groups from 1 to 3 days post-challenge. In contrast, cattle that received the FMDV trivalent mosaic vaccine (both single dose and boosted) were negative for viable virus isolation from serum samples though out the duration of the experiment (FIG. 18B).

### Viral Shedding (Detection of Infectious Virus)

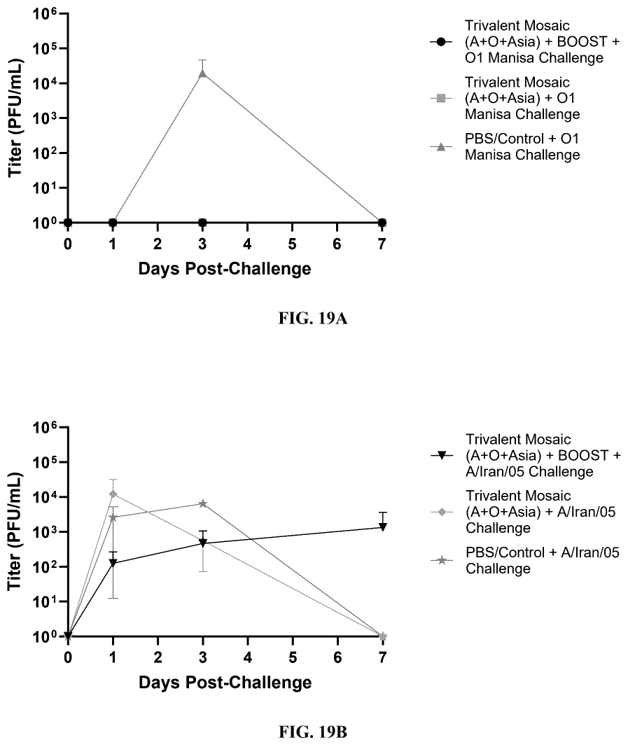

Isolation of viable virus from nasal swabs was titrated on BHK-21 αVβ6 monolayers under a tragacanth overlay and stained with crystal violet at 24 hours post infection (FIG. 19A and FIG. 19B). FMDV O1/Manisa challenge virus was isolated from serum samples of all the animals in control groups from 3 days post-challenge. In contrast, cattle that received the FMDV trivalent mosaic vaccine (both single dose and boosted) were negative for viable virus isolation from nasal swab samples throughout the duration of the experiment (FIG. 19A). FMDV A/Iran/05 challenge virus was isolated from serum samples of all the animals in control groups from 1 to 3 days post-challenge. Cattle that received the FMDV trivalent mosaic vaccine (both single dose and boosted) also showed viable FMDV A/Iran/05 isolated from nasal swab samples after challenge (FIG. 19B).

### Serum Viremia and Viral Shedding (Detection of Viral RNA)

In addition, FMDV RNA was detected by qRT-PCR in serum and nasal swab samples. Briefly, 50 μl of each sample (sera or nasal swab) for each cow was transferred to 96-well plates (King Fisher) containing 150 μl of lysis/binding solution. RNA was then extracted using MagMax-96 viral RNA isolation kit (Ambion) on a King Fisher-96 magnetic particle processor (Thermo Electron Corp.). After an initial 5-min lysis/binding step, the RNA samples underwent a series of four washing steps, a drying step, and a final elution step. RNA was eluted in a final volume of 25 μl. At each of the above steps, RNA was magnetically bound to the beads contained in the lysis/binding solution and was transferred to the different extraction solutions. RNA extracted from all the previous described samples was analyzed in duplicate by rRT-PCR using 2.5 μl of RNA on the QuantStudio 6 with the AgPath-ID one step RT-PCR kit (applied biosystems). Cycle threshold values were converted into log 10 FMDV genome copy numbers (GCN) per mL (log 10 GCN/mL), by use of standard curves based on analysis of 10-fold dilutions of in-vitro synthesized FMDV RNA.

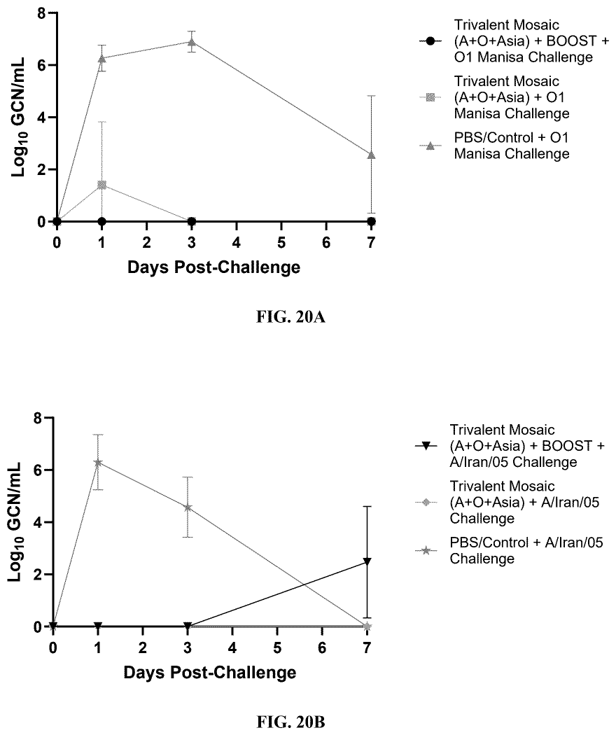

FMDV viral RNA was detected in the serum from control, PBS vaccinated animals challenged with FMDV O1/Manisa at 1, 3, and 7 DPC (FIG. 20A and FIG. 20B). Animals vaccinated with the FMDV trivalent mosaic vaccine (single dose) and challenged with FMDV O1/Manisa did show viral RNA present in the serum 24 hours after challenge lasting for only 1 day. Animals that received this trivalent vaccine in addition to a booster dose at 21 days post-vaccination did show viral RNA in the serum at any point following challenge with virulent FMDV O1/Manisa (FIG. 20A). Animals vaccinated with the FMDV trivalent mosaic vaccine (single dose) and challenged with FMDV A/Iran/05 did not show any viral RNA in nasal swab following challenge. Interestingly 2/3 animals that received this trivalent vaccine in addition to a booster dose at 21 days post-vaccination did show viral RNA in the nasal swab samples at 7 dpc (FIG. 20B).

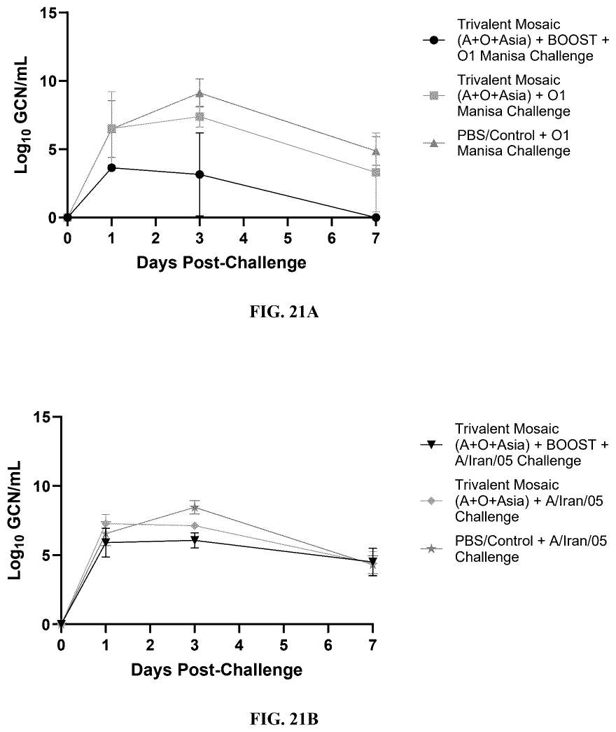

FMDV viral RNA was detected in the nasal swab samples from control, PBS vaccinated animals challenged with FMDV O1/Manisa at 1, 3, and 7 DPC (FIG. 21A and FIG. 21B). Animals vaccinated with the FMDV trivalent mosaic vaccine (single and booster) and challenged with FMDV O1/Manisa did show viral RNA present in the nasal swabs after challenge however, those animals that received the booster dose, showed a several log reduction in viral RNA (FIG. 21A). Likewise, animals vaccinated with the FMDV trivalent mosaic vaccine (single and booster) and challenged with FMDV A/Iran/05 did show viral RNA present in the nasal swabs after challenge. Animals receiving the booster vaccine dose, did show a 1-2 log reduction in viral RNA compared to the animals receiving only the single vaccine dose (FIG. 21B).

### Analysis of Hematological Parameters

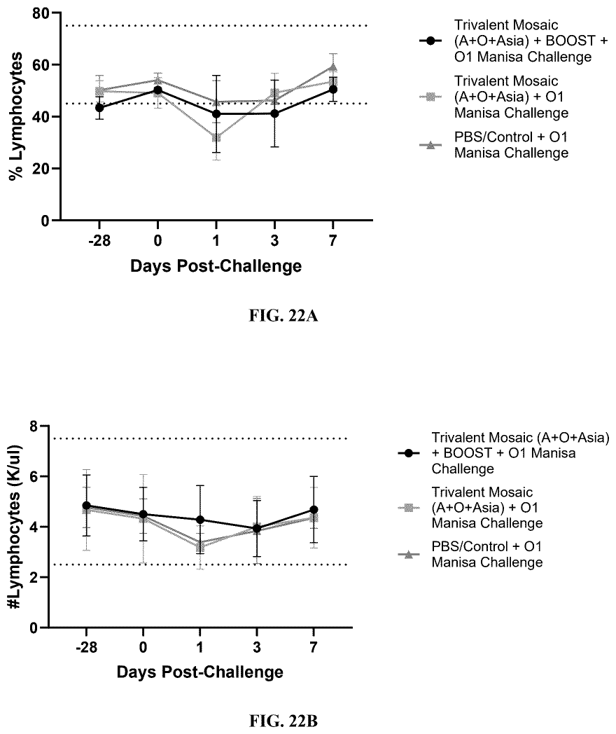

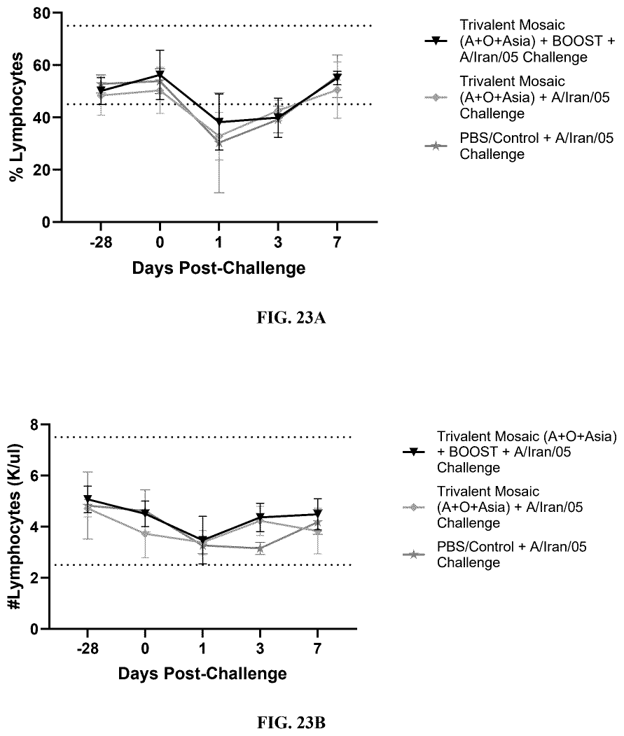

White blood cell subpopulations, specifically lymphocyte populations (FIG. 22A, FIG. 22B, FIG. 23A, and FIG. 23B) were investigated. For all animals, whole blood samples were collected in EDTA tubes and differential blood counts were conducted using a Hema Vet 950FS (Drew Scientific), according to manufacturer's instructions. Animals receiving the FMDV trivalent mosaic vaccine (single dose) and control mock-vaccinated animals challenged with FMDV 01/Manisa showed lymphocytopenia at 24 hours post-challenge. However, animals that received the FMDV trivalent vaccine in addition to a booster dose at 21 days post-vaccination, did not display a decrease in lymphocyte population following challenge with virulent FMDV O1/Manisa. All animals, regardless of vaccination status, challenged with FMDV A/Iran/05 showed a transient decrease in lymphocyte populations 24 hours post-challenge, but returned to normal range by three days post-challenge.

### Virus Neutralization Assay

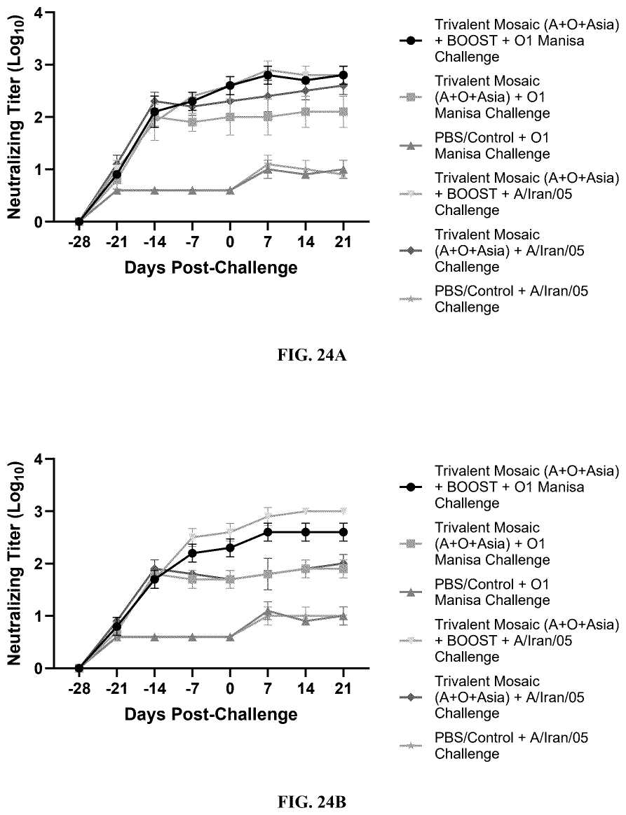

Neutralizing antibody titers against FMDV vaccine strain Asia Mosaic 2.1 and against FMDV wild type strain Asia Shamir were determined by the virus neutralization test method described in the OIE Terrestrial Manual (OIE, 2015) in 96-well plates of BHK αvβ6 cells, on serum samples collected at days 0, 7, 14, 21, 28, 35, 42, and 49. SVN titers were calculated according to the Spearman-Kärber method and expressed as log₁₀ of the reciprocal of the final serum dilution that neutralized 100 TCID₅₀ of the respective serotype Asia FMDV in 50% of the wells. The assay lower limit of detection was 0.6 log 10. All animals immunized with the trivalent Mosaic vaccine developed serum virus neutralizing antibodies to both Asia Mosaic 2.1 (FIG. 24A) and the wild type FMDV strain Asia Shamir by 7 dpv (FIG. 24B), reaching peak titers at 14 days post prime vaccination in the groups that were immunized once or by 14 days post boost immunization in the groups that were vaccinated twice (FIG. 24A (Asia Mosaic 2.1) and FIG. 24B (Asia Shamir)). Even though these animals were not challenged against FMDV serotype Asia, they all reached neutralizing titers of >1.8 at the time of challenge, which are considered protective according to the OIE Terrestrial Manual (OIE, 2015). As expected, none of the unvaccinated control animals had FMDV neutralizing titers on any day prior or at the day of challenge.

While the invention has been described with reference to details of the illustrated embodiments, these details are not intended to limit the scope of the invention as defined in the appended claims. The embodiment of the invention in which exclusive property or privilege is claimed is defined as follows:

What is claimed is:

1. A synthetic polypeptide comprising an amino acid sequence at least 95% identical to SEQ ID NO: 2, SEQ ID NO: 4, SEQ ID NO: 6, or SEQ ID NO: 8.

2. The synthetic polypeptide of claim 1, comprising the amino acid sequence of SEQ ID NO: 2, SEQ ID NO: 4, SEQ ID NO: 6, or SEQ ID NO: 8.

3. The synthetic polypeptide of claim 1 or claim 2, further comprising a pharmaceutically acceptable carrier.

4. The synthetic polypeptide of claim 1, comprising a first and a second synthetic polypeptide comprising a first synthetic polypeptide comprising an amino acid sequence at least 95% identical to SEQ ID NO: 2, a second synthetic polypeptide comprising an amino acid sequence at least 95% identical to SEQ ID NO: 4, and optionally a pharmaceutically acceptable carrier.

5. The synthetic polypeptide of claim 4, further comprising an adjuvant.

6. A recombinant foot-and-mouth disease virus (FMDV) comprising a synthetic polypeptide having an amino acid sequence at least 95% identical to SEQ ID NO: 2, SEQ ID NO: 4, SEQ ID NO: 6, or SEQ ID NO: 8.

7. The recombinant FMDV of claim 6, comprising a synthetic polypeptide comprising the amino acid sequence of SEQ ID NO: 2, SEQ ID NO: 4, SEQ ID NO: 6, or SEQ ID NO: 8.

8. The recombinant FMDV of claim 6, comprising a first and a second recombinant FMDV, wherein the first recombinant FMDV comprises a synthetic polypeptide at least 95% identical to the amino acid sequence of SEQ ID NO: 2, and the second recombinant FMDV comprises a synthetic polypeptide at least 95% identical to the amino acid sequence of SEQ ID NO: 4, and further optionally comprising a pharmaceutically acceptable carrier.

9. An isolated nucleic acid molecule encoding the synthetic polypeptide of claim 1.

10. The isolated nucleic acid molecule of claim 9, comprising the nucleotide sequence of SEQ ID NO: 1, SEQ ID NO: 3, SEQ ID NO: 5, or SEQ ID NO:7.

11. A vector comprising the isolated nucleic acid molecule of claim 10.

12. A method of eliciting an immune response against serotype Asia foot-and-mouth disease virus (FMDV) in a subject, comprising administering to the subject a composition comprising the synthetic polypeptide at least 95% identical to SEQ ID NO: 2, SEQ ID NO: 4, SEQ ID NO: 6, or SEQ ID NO: 8, thereby eliciting an immune response to serotype Asia FMDV.

13. The method of claim 12, wherein the synthetic polypeptide comprises SEQ ID NO: 2, SEQ ID NO: 4, SEQ ID NO: 6, or SEQ ID NO: 8.

14. The method of claim 12, wherein the composition further comprises a pharmaceutically acceptable carrier, an adjuvant, or both a pharmaceutically acceptable carrier and an adjuvant.

15. The method of claim 12, comprising administering to the subject a first composition comprising a synthetic polypeptide at least 98% identical to the amino acid sequence of SEQ ID NO: 2 and a second composition comprising a second synthetic polypeptide at least 95% identical to the amino acid sequence of SEQ ID NO: 4.

16. The method of claim 12, wherein the subject is a cow or a pig.


SEQUENCE LISTING (8 sequences)

SEQ ID NO: 1 - DNA, 2193 bp, synthetic construct
  source 1..2193: mol_type = other DNA
ggggccggccaatccagcccggcgactgggtcacagaaccaatcaggcaacactggaagcatcatcaacaactactacatgcagcagtaccagaactccatggacacacaacttggtgacaacgctatcagcggaggctccaacgaaggttccaccgacaccacctccacacacacaaacaacacccaaaacaacgattggttttcacgcctggctagttccgccttcagcggactgtttggcgcgcttctggctgacaagaaaacagaagagacaactttgcttgaagaccgcattctcaccaccaggaacggccacacgacgtcgacgacacagtcgagcgttggcgtgacatacggttacgctgtggctgaggacgcggtgtcagggcctaacacctcaggcctggagacccgtgttcaacaagcggaacggttcttcaaaaagcacttgtttgactggacaccgaatttggcatttggacactgtcactacctggaacttcccactgaacacaaaggcgtgtacggcagcctcatggactcgtacgcgtacatgaggaacgggtgggacatagaggtgactgctgttggaaaccagttcaacggcggttgtctccttgtcgcacttgtgccagagttgaagagccttgacacacggcagaagtaccaactgactctcttcccccaccagttcatcaacccacgcaccaacatgacggcccacatcaacgtgccctttgtgggtgttaacaggtatgaccagtacgcgctccacaagccgtggacgctcgttgtgatggtggtggccccactcaccgtcaagactggtggttctgaacaaatcaaggtttacatgaatgcagcaccaacctacgtgcacgtggcaggggagctgccctcgaaagaggggatagtacccgttgcgtgtgcggacggttatggcaacatggtgaccaccgacccgaagactgctgacccagtctacgggaaagtgttcaaccccccccggacaaatcttcccgggcgcttcacaaacttccttgatgttgcggaggcatgtccaaccttcctccgcttcggagaagtaccatttgtgaagacagtgaactctggtgaccgtttgctggccaagtttgatgtctcgctcgctgcggggcacatgtccaacacctacttggccggcttggcacagtactacacacagtacagtggcaccatgaacgttcacttcatgttcaccgggcccacggacgccaaagcccggtacatggtggcctacatccctcccggcatgacaccgcccacggaccctgagcgtgctgcacactgcattcactctgagtgggacactggtcttaactccaagttcaccttctccataccctacctctctgctgctgactacgcatacaccgcttctgacacggcggagaccacaagtgtgcaaggatgggtgtgcatctaccagatcacccacggcaaggctgaaggagacgcactggtcgtttctgtcagcgccggcaaagactttgagttccgcctgcccgttgacgcgcgccggcaaaccaccactaccggcgagtcagcggacccggtcacaaccacggttgagaactacggaggagaaactcagacagccagacggcttcacactgacgttgccttcgttcttgacaggtttgtgaaactcactgcacccaagaacattcagactcttgacctcatgcagatcccctcacacacactggttggagcgctgcttcggtctgcgacgtactacttctcagacctggaggttgcgctcgtccacacaggcccggtcacctgggtgcccaacggctcgcccaaggatgctctggacaaccagaccaacccaactgcctaccagaagcaacccatcacccgcctggcactcccctacaccgccccccaccgtgtgctggcaacagtgtacaacgggaagacaacgtacggggaaacaacctcacggcgtggtgacatggcagcccttgcacaaaggctgagcgggcggctgcccacttccttcaactacggcgctgtgaaggctgaaaccatcactgagcttttgatccgcatgaaacgcgcggagacatactgccccaggcctttgctggctcttgacaccactcaagaccgccgtaagcaggagatcattgcacctgagaagcag

SEQ ID NO: 2 - protein, 731 aa, synthetic construct
GAGQSSPATGSQNQSGNTGSIINNYYMQQYQNSMDTQLGDNAISGGSNEGSTDTTSTHTNNTQNNDWFSRLASSAFSGLFGALLADKKTEETTLLEDRILTTRNGHTTSTTQSSVGVTYGYAVAEDAVSGPNTSGLETRVQQAERFFKKHLFDWTPNLAFGHCHYLELPTEHKGVYGSLMDSYAYMRNGWDIEVTAVGNQFNGGCLLVALVPELKSLDTRQKYQLTLFPHQFINPRTNMTAHINVPFVGVNRYDQYALHKPWTLVVMVVAPLTVKTGGSEQIKVYMNAAPTYVHVAGELPSKEGIVPVACADGYGNMVTTDPKTADPVYGKVFNPPRTNLPGRFTNFLDVAEACPTFLRFGEVPFVKTVNSGDRLLAKFDVSLAAGHMSNTYLAGLAQYYTQYSGTMNVHFMFTGPTDAKARYMVAYIPPGMTPPTDPERAAHCIHSEWDTGLNSKFTFSIPYLSAADYAYTASDTAETTSVQGWVCIYQITHGKAEGDALVVSVSAGKDFEFRLPVDARRQTTTTGESADPVTTTVENYGGETQTARRLHTDVAFVLDRFVKLTAPKNIQTLDLMQIPSHTLVGALLRSATYYFSDLEVALVHTGPVTWVPNGSPKDALDNQTNPTAYQKQPITRLALPYTAPHRVLATVYNGKTTYGETTSRRGDMAALAQRLSGRLPTSFNYGAVKAETITELLIRMKRAETYCPRPLLALDTTQDRRKQEIIAPEKQ

SEQ ID NO: 3 - DNA, 2193 bp, synthetic construct
  source 1..2193: mol_type = other DNA
ggggccggccaatccagcccggcgactgggtcacagaaccaatcaggcaacactggaagcatcatcaacaactactacatgcagcagtaccagaactccatggacacacaacttggtgacaatgctatcagcggaggctccaacgaaggttccaccgacaccacctccacacacacaaccaacacccaaaacaacgattggttttcacgcctggctagttccgccttcaccggactgtttggcgcgcttctggctgacaagaaaacagaagaggcaactttgcttgaagaccgcattctcaccaccaggaacggccacacgacgtcgacgacacagtcgagcgttggcgtgacatacggttacgctacggctgaggacgcggtgtcagggcctaacacctcaggcctggagacccgtgttacacacgcggaacggttcttcaaaaagcacttgtttgactggacaccgaatttgtcatttggacactgtcactacctggaacttccctctgaacacaaaggcgtgtttggcagcctcatgagctcgtacgcgtacatgaggaacgggtgggacgttgaggtgactgctgttggaaaccagttcaacggcggttgtctccttgtcgcacttgtgccagagttgaaggaacttgacacacggcagaagtaccaactgactctcttcccccaccagttcatcaacccacgcaccaacatgacggcccacatcaacgtgccctacgtgggtgttaacaggtatgaccagtacgagctccacaagccgtggacgctcgttgtgatggtggtggccccactcaccgtcaagactggtggttctgaacaaatcaaggtttacatgaatgcagcaccaacccacgtgcacgtggcaggggagctgccctcgaaagaggggatagtacccgttgcgtgtgtggacggttatggcaacatggtgaccaccgacccgaagactgctgacccagtctacgggaaagtgtctaaccccccccggacaagcttccccgggcgcttcacaaactttcttgatgttgcggaggcatgtccaaccttcctccgcttcggagaagtaccatttgtgaagacagtgaactctggtgaccgtttgctggccaagtttgatgtctcgctcgctgcggggcacttgtccaacacctacttggccggcttggcacagtactacacacagtacagtggcaccatgaacattcacttcatgttcaccgggcccacggacgccaaagcccggtacatggtggcctacgtccctcccggcatgacaccgcccacggaacctgagcgtgctgcacactgcattcactctgagtgggacactggtcttaactccaagttcaccttctccataccctacctctctgctgctgactacgcatacaccgcttctgacgtggcggagaccacaagtgtgcaaggatgggtgtgcatctaccagatcacccacggcaaggctgaaggagacgcactggtcgtttctgtcagcgccggcaaagactttgagttccgcctgcccgttgacgcgcgccaacaaaccaccactaccggcgagtcagcggacccggtcacaaccacggttgagaactacggaggagaaactcaggcagccagacggcttcacactgacgttggtttcgttcttgacaggtttgtgagactcactcagcccaaggccacccagactcttgacctcatgcagatccccccatacacactggttggagcgctgcttcggtctgcgacgtactacttctcagacctggaggttgcgctcgtccacamaggctcagtcacctgggtgcccaacggcgcgcccaaggatgctctggacaaccacaccaacccaactgcctaccagaagaaacccatcacccgcctggcactcccctacaccgccccccaccgtgtgctgtcaacagtgtacaacgggaagacaacgtacgggacacaacccacgcggcgtggtgaccttgcagttcttgcacaaagggtgagcaaccggctgcccacttccttcaactacggcgctgtgaaggctgacaccatcactgagcttttgatccgcatgaaacgcgcggagacatactgccccaggcctttgctggctcttgacaccactcacgaccgccgtaagcaggagatcattgcacctgagaagcag

SEQ ID NO: 4 - protein, 731 aa, synthetic construct
GAGQSSPATGSQNQSGNTGSIINNYYMQQYQNSMDTQLGDNAISGGSNEGSTDTTSTHTTNTQNNDWFSRLASSAFTGLFGALLADKKTEEATLLEDRILTTRNGHTTSTTQSSVGVTYGYATAEDAVSGPNTSGLETRVTHAERFFKKHLFDWTPNLSFGHCHYLELPSEHKGVFGSLMSSYAYMRNGWDVEVTAVGNQFNGGCLLVALVPELKELDTRQKYQLTLFPHQFINPRTNMTAHINVPYVGVNRYDQYELHKPWTLVVMVVAPLTVKTGGSEQIKVYMNAAPTHVHVAGELPSKEGIVPVACVDGYGNMVTTDPKTADPVYGKVSNPPRTSFPGRFTNFLDVAEACPTFLRFGEVPFVKTVNSGDRLLAKFDVSLAAGHLSNTYLAGLAQYYTQYSGTMNIHFMFTGPTDAKARYMVAYVPPGMTPPTEPERAAHCIHSEWDTGLNSKFTFSIPYLSAADYAYTASDVAETTSVQGWVCIYQITHGKAEGDALVVSVSAGKDFEFRLPVDARQQTTTTGESADPVTTTVENYGGETQAARRLHTDVGFVLDRFVRLTQPKATQTLDLMQIPPYTLVGALLRSATYYFSDLEVALVHXGSVTWVPNGAPKDALDNHTNPTAYQKKPITRLALPYTAPHRVLSTVYNGKTTYGTQPTRRGDLAVLAQRVSNRLPTSFNYGAVKADTITELLIRMKRAETYCPRPLLALDTTHDRRKQEIIAPEKQ

SEQ ID NO: 5 - DNA, 2193 bp, synthetic construct
  source 1..2193: mol_type = other DNA
ggggccggccaatccagcccggcgactgggtcacagaaccaatcaggcaacactggaagcatcatcaacaactactacatgcagcagtaccagaactccatggacacacaacttggtgacaacgctatcagcggaggctccaacgaaggttccaccgacaccacctccacacacacaaacaacacccaaaacaacgattggttttcacgcctggctagttccgccttcagcggactgtttggcgcgcttctggctgacaagaaaacagaagagacaactttgcttgaagaccgcattctcaccaccaggaacggccacacgacgtcgacgacacagtcgagcgttggcgtgacatacggttacgctgtggctgaggacgcggtgtcagggcctaacacctcaggcctggagacccgtgttcaacaagcggaacggttcttcaaaaagcacttgtttgactggacaccgtatttggcatttggacactgtcactacctggaacttcccactgaacacaaaggcgtgtacggcagcctcatggactcgtacgcgtacatgaggaacgggtgggacatagaggtgactgctgttggaaaccagttcaacggcggttgtctccttgtcgcacttgtgccagagttgaagagccttgacacacggcagaagtaccaactgactctcttcccccaccagttcatcaacccacgcaccaacatgacggcccacatcaacgtgccctttgtgggtgttaacaggtatgaccagtacgcgctccacaagccgtggacgctcgttgtgatggtggtggccccactcaccgtcaagactggtggttctgaacaaatcaaggtttacatgaatgcagcaccaacctacgtgcacgtggcaggggagctgccctcgaaagaggggatagtacccgttgcgtgtgcggacggttatggcaacatggtgaccaccgacccgaagactgctgacccagtctacgggaaagtgttcaaccccccccggacaaatcttcccgggcgcttcacaaacttccttgatgttgcggaggcatgtccaaccttcctccgcttcggagaagtaccatttgtgaagacagtgaactctggtgaccgtttgctggccaagtttgatgtctcgctcgctgcggggcacatgtccaacacctacttggccggcttggcacagtactacacacagtacagtggcaccatgaacgttcacttcatgttcaccgggcccacggacgccaaagcccggtacatggtggcctacatccctcccggcatgacaccgcccacggaccctgagcgtgctgcacactgcattcactctgagtgggacactggtcttaactccaagttcaccttctccataccctacctctctgctgctgactacgcatacaccgcttctgacacggcggagaccacaagtgtgcaaggatgggtgtgcatctaccagatcacccacggcaaggctgaaggagacgcactggtcgtttctgtcagcgccggcaaagactttgagttccgcctgcccgttgacgcgcgccggcaaaccaccactaccggcgagtcagcggacccggtcacaaccacggttgagaactacggaggagaaactcagacagccagacggcttcacactgacgttgccttcgttcttgacaggtttgtgaaactcactgcacccaagaacattcagactcttgacctcatgcagatcccctcacacacactggttggagcgctgcttcggtctgcgacgtactacttctcagacctggaggttgcgctcgtccacacaggcccggtcacctgggtgcccaacggctcgcccaaggatgctctggacaaccagaccaacccaactgcctaccagaagcaacccatcacccgcctggcactcccctacaccgccccccaccgtgtgctggcaacagtgtacaacgggaagacaacgtacggggaaacaacctcacggcgtggtgacatggcagcccttgcacaaaggctgagcgggcggctgcccacttccttcaactacggcgctgtgaaggctgaaaccatcactgagcttttgatccgcatgaaacgcgcggagacatactgccccaggcctttgctggctcttgacaccactcaagaccgccgtaagcaggagatcattgcacctgagaagcag

SEQ ID NO: 6 - protein, 731 aa, synthetic construct
GAGQSSPATGSQNQSGNTGSIINNYYMQQYQNSMDTQLGDNAISGGSNEGSTDTTSTHTNNTQNNDWFSRLASSAFSGLFGALLADKKTEETTLLEDRILTTRNGHTTSTTQSSVGVTYGYAVAEDAVSGPNTSGLETRVQQAERFFKKHLFDWTPYLAFGHCHYLELPTEHKGVYGSLMDSYAYMRNGWDIEVTAVGNQFNGGCLLVALVPELKSLDTRQKYQLTLFPHQFINPRTNMTAHINVPFVGVNRYDQYALHKPWTLVVMVVAPLTVKTGGSEQIKVYMNAAPTYVHVAGELPSKEGIVPVACADGYGNMVTTDPKTADPVYGKVFNPPRTNLPGRFTNFLDVAEACPTFLRFGEVPFVKTVNSGDRLLAKFDVSLAAGHMSNTYLAGLAQYYTQYSGTMNVHFMFTGPTDAKARYMVAYIPPGMTPPTDPERAAHCIHSEWDTGLNSKFTFSIPYLSAADYAYTASDTAETTSVQGWVCIYQITHGKAEGDALVVSVSAGKDFEFRLPVDARRQTTTTGESADPVTTTVENYGGETQTARRLHTDVAFVLDRFVKLTAPKNIQTLDLMQIPSHTLVGALLRSATYYFSDLEVALVHTGPVTWVPNGSPKDALDNQTNPTAYQKQPITRLALPYTAPHRVLATVYNGKTTYGETTSRRGDMAALAQRLSGRLPTSFNYGAVKAETITELLIRMKRAETYCPRPLLALDTTQDRRKQEIIAPEKQ

SEQ ID NO: 7 - DNA, 2193 bp, synthetic construct
  source 1..2193: mol_type = other DNA
ggggccggccaatccagcccggcgactgggtcacagaaccaatcaggcaacactggaagcatcatcaacaactactacatgcagcagtaccagaactccatggacacacaacttggtgacaacgctatcagcggaggctccaacgaaagttccaccgacaccacctccacacacacaaccaacacccaaaacaacgattggttttcacgcctggctagttccgccttcaccggactgtttggcgcgcttctggctgacaagaaaacagaagaggcaactttgcttgaagaccgcattctcaccaccaggaacggccacacgacgtcgacgacacagtcgagcgttggcgtgacatacggttacgctacggctgaggacgcggtgtcagggcctaacacctcaggcctggagacccgtgttacacaagcggaacggttcttcaaaaagcacttgtttgactggacaccgaatttgtcatttggacactgtcactacctggaacttccctctgaacacaaaggcgtgtttggcagcctcatgagctcgtacgcgtacatgaggaacgggtgggacgttgaggtgactgctgttggaaaccagttcaacggcggttgtctccttgtcgcacttgtgccagagttgaaggaacttgacacacggcagaagtaccaactgactctcttcccccaccagttcatcaacccacgcaccaacatgacggcccacatcaacgtgccctacgtgggtgttaacaggtatgaccagtacgagctccacaagccgtggacgctcgttgtgatggtggtggccccactcaccgtcaagactggtggttctgaacaaatcaaggtttacatgaatgcagcaccaacccacgtgcacgtggcaggggagctgccctcgaaagaggggatagtacccgttgcgtgtgtggacggttatggcaacatggtgaccaccgacccgaagactgctgacccagtctacgggaaagtgtctaaccccccccggacaagcttccccgggcgcttcacaaacttccttgatgttgcggaggcatgtccaaccttcctccgcttcggagaagtaccatttttgaagacagtgaactctggtgaccgtttgctggccacgtttgatgtctcgctcgctgcggggcacttgtccaacacctacttggccggcttggcacagtactacacacagtacagtggcaccatgaacattcacttcatgttcaccgggcccacggacgccaaagcccggtacatggttgcctacgtccctcccggcatgacaccgcccacggaacctgagcgtgctgcacactgcattcactctgagtgggacactggtcttaactccaagttcaccttctccataccctacctctctgctgctgactacgcatacaccgcttctgacgtggcggagaccacaagtgtgcaaggatgggtgtgcatctaccagatcacccacggcaaggctgaaggagacgcactggtcgtttctgtcagcgccggcaaagactttgagttccgcctgcccgttgacgcgcgccaacaaaccaccactaccggcgagtcagcggacccggtcacaaccacggttgagaactacggaggagaaactcaggcagccagacggcttcacactgacgttggtttcgttcttgacaggtttgtgagactcactcagcccaaggccacccagactcttgacctcatgcagatccccccatacacactggttggagcgctgcttcggtctgcgacgtactacttctcagacctggaggttgcgctcgtccacacaggctcagtcacctgggtgcccaacggcgcgcccaaggatgctctggacaaccacaccaacccaactgcctaccagaagaaacccatcacccgcctggcactcccctacaccgccccccaccgtgtgctgtcaacagtgtacaacgggaagacaacgtacgggacacaacccacgcggcgtggtgaccttgcagttcttgcacaaagggtgagcaaccggctgcccacttccttcaactacggcgctgtgaaggctgacaccatcactgagcttttgatccgcatgaaacgcgcggagacatactgccccaggcctttgctggctcttgacaccactcacgaccgccgtaagcaggagatcattgcacctgagaagcag

SEQ ID NO: 8 - protein, 731 aa, synthetic construct
GAGQSSPATGSQNQSGNTGSIINNYYMQQYQNSMDTQLGDNAISGGSNESSTDTTSTHTTNTQNNDWFSRLASSAFTGLFGALLADKKTEEATLLEDRILTTRNGHTTSTTQSSVGVTYGYATAEDAVSGPNTSGLETRVTQAERFFKKHLFDWTPNLSFGHCHYLELPSEHKGVFGSLMSSYAYMRNGWDVEVTAVGNQFNGGCLLVALVPELKELDTRQKYQLTLFPHQFINPRTNMTAHINVPYVGVNRYDQYELHKPWTLVVMVVAPLTVKTGGSEQIKVYMNAAPTHVHVAGELPSKEGIVPVACVDGYGNMVTTDPKTADPVYGKVSNPPRTSFPGRFTNFLDVAEACPTFLRFGEVPFLKTVNSGDRLLATFDVSLAAGHLSNTYLAGLAQYYTQYSGTMNIHFMFTGPTDAKARYMVAYVPPGMTPPTEPERAAHCIHSEWDTGLNSKFTFSIPYLSAADYAYTASDVAETTSVQGWVCIYQITHGKAEGDALVVSVSAGKDFEFRLPVDARQQTTTTGESADPVTTTVENYGGETQAARRLHTDVGFVLDRFVRLTQPKATQTLDLMQIPPYTLVGALLRSATYYFSDLEVALVHTGSVTWVPNGAPKDALDNHTNPTAYQKKPITRLALPYTAPHRVLSTVYNGKTTYGTQPTRRGDLAVLAQRVSNRLPTSFNYGAVKADTITELLIRMKRAETYCPRPLLALDTTHDRRKQEIIAPEKQ